(19) United States
(12) Patent Application Publication
(10) Pub. No.: US 2026/0224532 A1
(43) Pub. Date: Aug. 6, 2026

(54) ESTROGEN RECEPTOR BETA LIGANDS FOR THE PREVENTION AND TREATMENT OF MULTIPLE SCLEROSIS (MS) AND OTHER DEMYELINATING, INFLAMMATORY AND NEURODEGENERATIVE DISEASES

(71) Applicants: The Board of Trustees of the University of Illinois, Urbana, IL (US); The Regents of the University of California, Oakland, CA (US)

(72) Inventors: John A. Katzenellenbogen, Urbana, IL (US); Sung Hoon Kim, Champaign, IL (US); Seema K. Tiwari-Woodruff, Riverside, CA (US)

(21) Appl. No.: 19/159,708

(22) PCT Filed: Mar. 7, 2024
(86) PCT No.: PCT/US2024/018837

§ 371 Date: Aug. 26, 2025

Related U.S. Application Data
(60) Provisional application No. 63/488,883, filed on Mar. 7, 2023.

(51) Int. Cl. A61K 31/416; A61P 25/00

(52) U.S. Cl. CPC A61K 31/416; A61P 25/00

(57) ABSTRACT

6- or 7-Substituted 2-(4-hydroxyphenyl)-2H-indazol-5-ol compounds are estrogen receptor beta ligands that have immunomodulatory properties and increase oligodendrocyte survival, differentiation, and remyelination. The compounds, compositions, and kits are useful in the treatment of multiple sclerosis and endometriosis.

### RELATED APPLICATIONS

This application claims priority to U.S. Provisional Application No. 63/488,883, filed Mar. 7, 2023, which is incorporated herein by reference in its entirety.

### STATEMENT REGARDING FEDERALLY SPONSORED RESEARCH OR DEVELOPMENT

This invention was made with government support under grant number R01 DK015556 awarded by the National Institutes of Health (NIH). The government has certain rights in the invention.

### TECHNICAL FIELD

The invention relates to estrogen receptor beta ligands and their use in the treatment of demyelinating diseases and endometriosis.

### BACKGROUND

Demyelinating diseases are characterized by damage to the myelin sheaths of the central nervous system. Once myelin sheaths are damaged, axons are left exposed and are unable to effectively transmit nerve impulses. Symptoms include vision loss, muscle weakness, muscle stiffness and spasms, loss of coordination, change in sensation, pain, and changes in bladder and bowel function.

Multiple sclerosis (MS) is the most common autoimmune demyelinating and neurodegenerative disease of the central nervous system (CNS). There is no known cause or cure for MS. Experimental autoimmune encephalomyelitis (EAE) recapitulates the inflammation, demyelination, and neurodegeneration observed in MS and is among the most common inducible animal model of MS. The EAE model has been used to develop many currently approved MS treatments, including interferon (IFN) B, glatiramer acetate, fingolimod, and the anti-CD20 monoclonal antibody, ocrelizumab. Although the approved therapeutics are effective at treating various symptoms and are able to attenuate inflammation, they cannot reverse or prevent neurodegeneration nor initiate remyelination.

Accumulating evidence indicates that estrogens are both neuroprotective and immunomodulatory, making them attractive candidates for the treatment of MS (Khalaj, 2016). Estrogens skew the inflammatory T helper (Th)1 response prevalent in MS towards an anti-inflammatory Th2 profile (Cua et al., 1995; Nicot, 2009). Furthermore, in preclinical studies, treatment with pregnancy levels of the placenta-derived estrogenic hormone estriol attenuated EAE disease severity (Jansson and Holmdahl, 1998; Kim et al., 1999). However, although they display immense potential for treating MS, endogenous estrogen therapy possesses several undesirable or deleterious side effects (Banks and Canfell, 2009). In addition to feminizing male recipients, treatment with endogenous estrogens increase the risk of developing breast and endometrial cancers in females (Banks and Canfell, 2009). Importantly, the carcinogenic effects of estrogens are mediated through estrogen receptor (ER)α and not ERβ, suggesting that therapies targeting specific ER subtypes may impart the benefit of estrogen treatment, while circumventing these side effects (Burns and Korach, 2012).

It is of critical importance to develop and provide new therapeutics that can trigger significant remyelination and offer neuroprotection as well as modulation of the immune system without unwanted side effects.

### SUMMARY

The present invention provides compounds or a pharmaceutically acceptable salt thereof and the methods and compositions disclosed herein for treating a demyelinating disease, for differentiating oligodendrocyte progenitor cells, or for promoting remyelination of demyelinated axons.

In one aspect, the invention provides compounds of formula (I), or pharmaceutically acceptable salts thereof

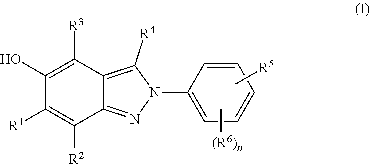

wherein:
R¹ and R² are independently hydrogen, halogen, C₁₋₄alkyl, C₁₋₄fluoroalkyl, C₂₋₄alkenyl, cyano, OH, —OC₁₋₄alkyl, or —OC₁₋₄fluoroalkyl, provided that at least one of R¹ and R² is not hydrogen;
R³ is hydrogen, halogen, or C₁₋₄alkyl;
R⁴ is hydrogen, halogen, C₁₋₄alkyl, C₁₋₄fluoroalkyl, or C₂₋₄alkenyl;
R⁵ is OH, wherein R⁵ is substituted at either the meta or para position;
R⁶, at each occurrence, is independently halogen, C₁₋₄alkyl, C₁₋₄fluoroalkyl, C₂₋₄alkenyl, cyano, OH, —OC₁₋₄alkyl, or —OC₁₋₄fluoroalkyl; and
n is 0, 1, or 2.

In another aspect, the invention provides a pharmaceutical composition comprising a compound of formula (I), or a pharmaceutically acceptable salt thereof, and a pharmaceutically acceptable carrier.

In another aspect, the invention provides a method of treating a demyelinating disease comprising administering, to a subject in need thereof, a therapeutically effective amount of the compound of formula (I), or a pharmaceutically acceptable salt or composition thereof.

In another aspect the invention provides a method of promoting remyelination of demyelinated axons comprising administering to a subject in need thereof a therapeutically effective amount of the compound of formula (I), or a pharmaceutically acceptable salt or composition thereof.

In another aspect the invention provides a method of differentiating oligodendrocyte progenitor cells comprising administering to a subject in need thereof a therapeutically effective amount of the compound of formula (I), or a pharmaceutically acceptable salt or composition thereof.

In another aspect, the invention provides a method of treating endometriosis comprising administering, to a subject in need thereof, a therapeutically effective amount of the compound of formula (I), or a pharmaceutically acceptable salt or composition thereof.

In another aspect, the invention provides a compound of formula (I), or a pharmaceutically acceptable salt or composition thereof, for use in the treatment of a demyelinating disease, or in the promotion of remyelination of demyelinated axons, or in the differentiation of oligodendrocyte progenitor cells, or in the treatment of endometriosis.

In another aspect, the invention provides the use of a compound of formula (I), or a pharmaceutically acceptable salt or composition thereof, in the manufacture of a medicament for the treatment of a demyelinating disease, or for the promotion of remyelination of demyelinated axons, or for the differentiation of oligodendrocyte progenitor cells, or for the treatment of endometriosis.

In another aspect, the invention provides a kit comprising a compound of formula (I), or a pharmaceutically acceptable salt or composition thereof, and instructions for use.

### BRIEF DESCRIPTION OF THE DRAWINGS

FIG. 1A-1D show the effects of treatment with the 7-substituted indazole compounds 1-5, 7, 9, and 11 on the expression of myelin basic protein in OLs (MBP⁺-OLs, top two panels), as well as the count of the total cells as an indication of cell proliferation/survival (DAPI nucleus stain, bottom two panels).

FIG. 2A shows the effects of treatment with the 6-substituted indazole compounds 17-20, on the expression of myelin basic protein in OLs (MBP⁺-OLs).

FIG. 2B shows the effects of treatment with the 6-substituted indazole compounds 17-20 on the count of the total cells as an indication of cell proliferation/survival (DAPI nucleus stain).

FIG. 3 shows results of a 2-hour single point pharmacokinetic (PK) study by either oral (PO) or subcutaneous (SC) dosing with compounds 7, 12, and 14.

FIG. 4 shows results of a 2-hour single point pharmacokinetic (PK) study by either oral (PO) or subcutaneous (SC) dosing with compound 17.

FIG. 5 shows the effects of compounds 5, 7, 12, and 14 on myelination in the cuprizone model. The height of each bar graph represents level of myelination assessed by the expression of MOG in the white matter tracks of the Corpus Callosum area quantified by immunohistochemical analysis (IHC) after staining with a MOG antibody. N: Normal diet, DM: Demyelinated status after 6 weeks cuprizone treatment, SC-Veh: Spontaneous remyelination after injection with matrix in saline solution only (without compound; recovery is only about 40%).

FIG. 6 shows the effect of compound 17 on myelination in the cuprizone model. The height of each bar graph represents level of myelination assessed by the expression of MOG in the white matter tracks of the Corpus Callosum area quantified by immunohistochemical analysis (IHC) after staining with a MOG antibody. N: Normal diet, DM: Demyelinated status after 6 weeks cuprizone treatment, SC-Veh: Spontaneous remyelination after injection with matrix in saline solution only (without compound; recovery is only about 40%).

FIG. 7 shows the effects of compounds 5 and 7 on uterine weight compared to vehicle in non-ovariectomized mice in the cuprizone model.

### DETAILED DESCRIPTION

### 1. Definitions

As described herein, compounds of the invention can optionally be substituted with one or more substituents, such as are illustrated generally above, or as exemplified by particular classes, subclasses, and species of the invention. As described herein, the variables in formula I encompass specific groups, such as, for example, alkyl and cycloalkyl. As one of ordinary skill in the art will recognize, combinations of substituents envisioned by this invention are those combinations that result in the formation of stable or chemically feasible compounds. The term “stable,” as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

Unless otherwise defined, all technical and scientific terms used herein have the same meaning as commonly understood by one of ordinary skill in the art. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkyl” as used herein, means a straight or branched chain saturated hydrocarbon. The term “C₁₋₄alkyl” means a straight or branched chain hydrocarbon containing from 1 to 4 carbon atoms. Representative examples of alkyl include, but are not limited to, methyl, ethyl, n-propyl, isopropyl, n-butyl, sec-butyl, isobutyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl,” as used herein, means a straight or branched, hydrocarbon chain containing at least one carbon-carbon double bond.

The term “alkylene,” as used herein, refers to a divalent group derived from a straight or branched chain saturated hydrocarbon. Representative examples of alkylene include, but are not limited to, —CH₂—, —CD₂-, —CH₂CH₂—, —C(CH₃)(H)—, —C(CH₃)(D)-, —CH₂CH₂CH₂—, —CH₂CH₂CH₂CH₂—, and —CH₂CH₂CH₂CH₂CH₂—.

The term “halogen” means a chlorine, bromine, iodine, or fluorine atom.

The term “haloalkyl,” as used herein, means an alkyl, as defined herein, in which one, two, three, four, five, six, or seven hydrogen atoms are replaced by halogen. For example, representative examples of haloalkyl include, but are not limited to, 2-fluoroethyl, difluoromethyl, trifluoromethyl, 2,2,2-trifluoroethyl, 2,2,2-trifluoro-1,1-dimethylethyl, and the like.

The term “fluoroalkyl,” as used herein, means an alkyl group, as defined herein, in which one, two, three, four, five, six, seven or eight hydrogen atoms are replaced by fluorine. Representative examples of fluoroalkyl include, but are not limited to, 2-fluoroethyl, 2,2,2-trifluoroethyl, trifluoromethyl, difluoromethyl, pentafluoroethyl, and trifluoropropyl such as 3,3,3-trifluoropropyl.

Terms such as “alkyl,” “cycloalkyl,” “alkylene,” “cycloalkylene,” etc. may be preceded by a designation indicating the number of atoms present in the group in a particular instance (e.g., “C₁₋₄alkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Thus, included within the scope of the invention are tautomers of compounds of formula I. The structures also include zwitterionic forms of the compounds or salts of formula I where appropriate.

The terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the regenerative cells, the type or extent of supplemental therapy used, ongoing disease process and type of treatment desired (e.g., aggressive vs. conventional treatment).

As used herein, “treat,” “treating” and the like means a slowing, stopping or reversing of progression of a disease or disorder when provided a composition described herein to an appropriate control subject. The term also means a reversing of the progression of such a disease or disorder to a point of eliminating or greatly reducing the cell proliferation. As such, “treating” means an application or administration of the compositions described herein to a subject, where the subject has a disease or a symptom of a disease, where the purpose is to cure, heal, alleviate, relieve, alter, remedy, ameliorate, improve or affect the disease or symptoms of the disease.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

As used herein, the terms “providing”, “administering,” “introducing,” are used interchangeably herein and refer to the placement of the compositions of the disclosure into a subject by a method or route which results in at least partial localization of the composition to a desired site. The compositions can be administered by any appropriate route which results in delivery to a desired location in the subject.

### 2. Compounds

A first aspect of the invention provides compounds or compositions of formula (I), or a pharmaceutically acceptable salt thereof, wherein R¹-R⁶ and n are as defined herein.

In the following, numbered embodiments of the invention are disclosed. The first embodiment is denoted E1, and subsequent embodiments are denoted E1.1, E2, E2.1, E2.2, E2.3, E3, etc.

E1. A compound of formula (I), or a pharmaceutically acceptable salt thereof,

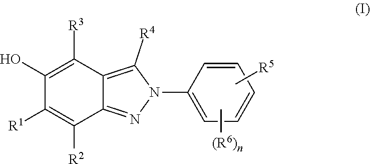

wherein:
R¹ and R² are independently hydrogen, halogen, C₁₋₄alkyl, C₁₋₄fluoroalkyl, C₂₋₄alkenyl, cyano, OH, —OC₁₋₄alkyl, or —OC₁₋₄fluoroalkyl, provided that at least one of R¹ and R² is not hydrogen;
R³ is hydrogen, halogen, or C₁₋₄alkyl;
R⁴ is hydrogen, halogen, C₁₋₄alkyl, C₁₋₄fluoroalkyl, or C₂₋₄alkenyl;
R⁵ is OH, wherein R⁵ is substituted at either the meta or para position;
R⁶, at each occurrence, is independently halogen, C₁₋₄alkyl, C₁₋₄fluoroalkyl, C₂₋₄alkenyl, cyano, OH, —OC₁₋₄alkyl, or —OC₁₋₄fluoroalkyl; and
n is 0, 1, or 2.

E1.1. The compound of E1, or a pharmaceutically acceptable salt thereof, wherein R¹ and R² are independently hydrogen, halogen, C₁₋₄alkyl, C₁₋₄fluoroalkyl, C₂₋₄alkenyl, cyano, —OC₁₋₄alkyl, or —OC₁₋₄fluoroalkyl, provided that at least one of R¹ and R² is not hydrogen.

E2. The compound of E1 or E1.1, or a pharmaceutically acceptable salt thereof, wherein R¹ is halogen, C₁₋₄alkyl, C₁₋₄fluoroalkyl, C₂₋₄alkenyl, cyano, —OC₁₋₄alkyl, or —OC₁₋₄fluoroalkyl.

E2.1. The compound of E2, or a pharmaceutically acceptable salt thereof, wherein R¹ is halogen.

E2.2. The compound of E2.1, or a pharmaceutically acceptable salt thereof, wherein R¹ is fluoro.

E2.3. The compound of E2.1, or a pharmaceutically acceptable salt thereof, wherein R¹ is choro.

E3. The compound of E1 or E1.1, or a pharmaceutically acceptable salt thereof, wherein R¹ is hydrogen.

E4. The compound of any of E1 or E2-E3, or a pharmaceutically acceptable salt thereof, wherein R² is halogen, C₁₋₄alkyl, C₁₋₄fluoroalkyl, C₂₋₄alkenyl, cyano, OH, —OC₁₋₄alkyl, or —OC₁₋₄fluoroalkyl.

E4.1. The compound of any of E1-E4, or a pharmaceutically acceptable salt thereof, wherein R² is halogen, C₁₋₄alkyl, C₁₋₄fluoroalkyl, C₂₋₄alkenyl, cyano, —OC₁₋₄alkyl, or —OC₁₋₄fluoroalkyl.

E4.2. The compound of E4.1, or a pharmaceutically acceptable salt thereof, wherein R² is halogen.

E4.3. The compound of E4.2, or a pharmaceutically acceptable salt thereof, wherein R² is fluoro.

E4.4. The compound of E4.2, or a pharmaceutically acceptable salt thereof, wherein R² is chloro.

E4.5. The compound of E4.2, or a pharmaceutically acceptable salt thereof, wherein R² is bromo.

E4.6. The compound of E4.1, or a pharmaceutically acceptable salt thereof, wherein R² is C₁₋₄alkyl.

E4.7. The compound of E4.6, or a pharmaceutically acceptable salt thereof, wherein R² is methyl.

E4.8. The compound of E4, or a pharmaceutically acceptable salt thereof, wherein R² is OH.

E5. The compound of any of E2-E2.3, or a pharmaceutically acceptable salt thereof, wherein R² is hydrogen.

E6. The compound of any of E1-E5, or a pharmaceutically acceptable salt thereof, wherein R³ is hydrogen.

E7. The compound of any of E1-E6, or a pharmaceutically acceptable salt thereof, wherein R⁴ is halogen.

E7.1. The compound of E7, or a pharmaceutically acceptable salt thereof, wherein R⁴ is fluoro.

E7.2. The compound of E7, or a pharmaceutically acceptable salt thereof, wherein R⁴ is chloro.

E7.3. The compound of E7, or a pharmaceutically acceptable salt thereof, wherein R⁴ is bromo.

E7.4. The compound of E7, or a pharmaceutically acceptable salt thereof, wherein R⁴ is iodo.

E8. The compound of any of E1-E6, or a pharmaceutically acceptable salt thereof, wherein R⁴ is hydrogen.

E9. The compound of any of E1-E8, or a pharmaceutically acceptable salt thereof, wherein R⁵ is substituted at the meta position, as shown in formula (I-A):

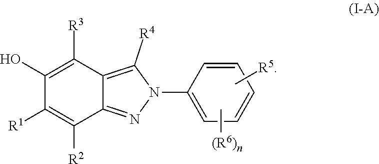

E10. The compound of any of E1-E8, or a pharmaceutically acceptable salt thereof, wherein R⁵ is substituted at the para position, as shown in formula (I-B):

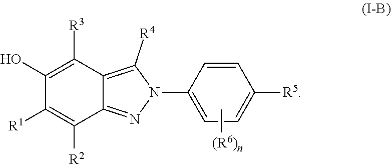

E11. The compound of any of E1-E10, or a pharmaceutically acceptable salt thereof, wherein n is 1.

E12. The compound of E11, or a pharmaceutically acceptable salt thereof, wherein the compound has formula (II):

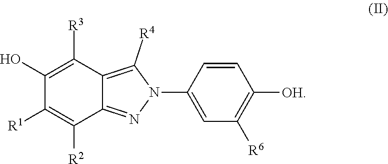

E13. The compound of E11, or a pharmaceutically acceptable salt thereof, wherein the compound has formula (III):

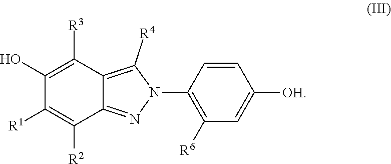

E14. The compound of any of E1-E13, or a pharmaceutically acceptable salt thereof, wherein R⁶, at each occurrence, is independently halogen or C₁₋₄alkyl.

E14.1. The compound of E14, or a pharmaceutically acceptable salt thereof, wherein R⁶, at each occurrence, is independently halogen (i.e., the halogen may be the same or different).

E14.2. The compound of E14.1, or a pharmaceutically acceptable salt thereof, wherein R⁶, at each occurrence, is fluoro.

E14.3. The compound of E14, or a pharmaceutically acceptable salt thereof, wherein R⁶, at each occurrence, is independently C₁₋₄alkyl (i.e., the alkyl may be the same or different).

E14.4. The compound of E14.3, or a pharmaceutically acceptable salt thereof, wherein R⁶, at each occurrence, is methyl.

E15. The compound of any of E1-E10, or a pharmaceutically acceptable salt thereof, wherein n is 0.

E16. The compound of E15, or a pharmaceutically acceptable salt thereof, wherein the compound has formula (IV):

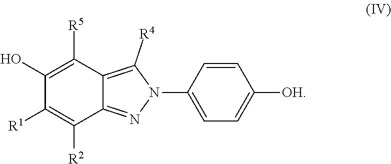

E17. The compound of E15, or a pharmaceutically acceptable salt thereof, wherein the compound has formula (V):

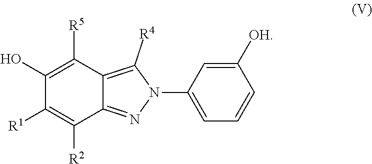

E18. The compound of E1 selected from the group consisting of:
7-bromo-2-(4-hydroxyphenyl)-2H-indazol-5-ol;
7-bromo-3-fluoro-2-(4-hydroxyphenyl)-2H-indazol-5-ol;
7-bromo-3-chloro-2-(4-hydroxyphenyl)-2H-indazol-5-ol;
3,7-dibromo-2-(4-hydroxyphenyl)-2H-indazol-5-ol;
7-bromo-2-(4-hydroxyphenyl)-3-iodo-2H-indazol-5-ol;
7-chloro-2-(4-hydroxyphenyl)-2H-indazol-5-ol;
3,7-dichloro-2-(4-hydroxyphenyl)-2H-indazol-5-ol;
7-fluoro-2-(4-hydroxyphenyl)-2H-indazol-5-ol;
3-chloro-7-fluoro-2-(4-hydroxyphenyl)-2H-indazol-5-ol;
7-methyl-2-(4-hydroxyphenyl)-2H-indazol-5-ol;
3-chloro-7-methyl-2-(4-hydroxyphenyl)-2H-indazol-5-ol;
7-bromo-3-chloro-2-(3-hydroxyphenyl)-2H-indazol-5-ol;
7-chloro-2-(3-fluoro-4-hydroxyphenyl)-2H-indazol-5-ol;
3,7-dichloro-2-(3-fluoro-4-hydroxyphenyl)-2H-indazol-5-ol;
7-chloro-2-(2-fluoro-4-hydroxyphenyl)-2H-indazol-5-ol;
3,7-dichloro-2-(2-fluoro-4-hydroxyphenyl)-2H-indazol-5-ol;
3-chloro-6-fluoro-2-(4-hydroxyphenyl)-2H-indazol-5-ol;
3-chloro-6-fluoro-2-(4-hydroxy-2-methylphenyl)-2H-indazol-5-ol;
3,6-dichloro-2-(4-hydroxyphenyl)-2H-indazol-5-ol;
3,6-dichloro-2-(4-hydroxy-2-methylphenyl)-2H-indazol-5-ol;
3-chloro-2-(4-hydroxyphenyl)-2H-indazole-5,7-diol; and
3-chloro-2-(4-hydroxy-2-methylphenyl)-2H-indazole-5,7-diol;
   or a pharmaceutically acceptable salt thereof.

E19. A pharmaceutical composition comprising the compound of any of E1-E18, or a pharmaceutically acceptable salt thereof, and a pharmaceutically acceptable carrier.

E20. A method of treating a demyelinating disease comprising, administering to a subject in need thereof, a therapeutically effective amount of the compound of any of E1-E18, or a pharmaceutically acceptable salt thereof, or the pharmaceutical composition of E19.

E21. The method of E20, wherein the demyelinating disease is multiple sclerosis.

E22. The method of E21, wherein the multiple sclerosis is primary progressive multiple sclerosis, relapsing-remitting multiple sclerosis, secondary progressive multiple sclerosis, or progressive relapsing multiple sclerosis.

E23. A method of promoting remyelination of demyelinated axons comprising administering to a subject in need thereof a therapeutically effective amount of the compound of any of E1-E18, or a pharmaceutically acceptable salt thereof, or the pharmaceutical composition of E19.

E24. A method of differentiating oligodendrocyte progenitor cells comprising administering to a subject in need thereof a therapeutically effective amount of the compound of any of E1-E18, or a pharmaceutically acceptable salt thereof, or the pharmaceutical composition of E19.

E25. A method of treating endometriosis comprising administering, to a subject in need thereof, a therapeutically effective amount of the compound of any of E1-E18, or a pharmaceutically acceptable salt thereof, or the pharmaceutical composition of E19.

E26. The compound of any of E1-E18, or a pharmaceutically acceptable salt thereof, or the pharmaceutical composition of E19, for use in the treatment of a demyelinating disease, or in the promotion of remyelination of demyelinated axons, or in the differentiation of oligodendrocyte progenitor cells, or in the treatment of endometriosis.

E27 The use of the compound of any of E1-E18, or a pharmaceutically acceptable salt thereof, or the pharmaceutical composition of E19, in the manufacture of a medicament for the treatment of a demyelinating disease, or for the promotion of remyelination of demyelinated axons, or for the differentiation of oligodendrocyte progenitor cells, or for the treatment of endometriosis.

E28. A kit comprising the compound of any of E1-E18, or a pharmaceutically acceptable salt thereof, or the pharmaceutical composition of E19, and instructions for use thereof.

Certain compounds may exist in one or more particular geometric, optical, enantiomeric, diastereomeric, epimeric, atropic, stereoisomer, tautomeric, conformational, or anomeric forms, including but not limited to, cis- and trans-forms; E- and Z-forms; c-, t-, and r-forms; endo- and exo-forms; R—, S—, and meso-forms; D- and L-forms; d- and 1-forms; (+) and ( ) forms; keto-, enol-, and enolate-forms; syn- and anti-forms; synclinal- and anticlinal-forms; a- and β-forms; axial and equatorial forms; boat-, chair-, twist-, envelope-, and half chair-forms; and combinations thereof, hereinafter collectively referred to as “isomers” (or “isomeric forms”).

Compounds may be prepared in racemic form or as individual enantiomers or diastereomers by either stereospecific synthesis or by resolution. The compounds may, for example, be resolved into their component enantiomers or diastereomers by standard techniques, such as the formation of stereoisomeric pairs by salt formation with an optically active base, followed by fractional crystallization and regeneration of the free acid. The compounds may also be resolved by formation of stereoisomeric esters or amides, followed by chromatographic separation and removal of the chiral auxiliary. Alternatively, the compounds may be resolved using a chiral HPLC column. The enantiomers also may be obtained from kinetic resolution of the racemate of corresponding esters using lipase enzymes.

Exemplary tautomeric forms include, for example, the following tautomeric pairs: keto/enol and imine/enamine.

In the compounds of formula (I), and any subformulas, any “hydrogen” or “H,” whether explicitly recited or implicit in the structure, encompasses hydrogen isotopes ¹H (protium) and ²H (deuterium).

In another embodiment, the compounds include isotope-labelled forms. An isotope-labelled form of a compound is identical to the compound apart from the fact that one or more atoms of the compound have been replaced by an atom or atoms having an atomic mass or mass number which differs from the atomic mass or mass number of the atom which usually occurs in greater natural abundance. Examples of isotopes which are readily commercially available and which can be incorporated into a compound by well-known methods include isotopes of hydrogen, carbon, nitrogen, oxygen, fluorine and chlorine, for example ²H, ³H, ¹³C, ¹⁴C, ¹⁵N, ¹⁸O, ¹⁷O, ¹⁸F and ³⁶Cl. Isotopically-labeled compounds of formula (I) can generally be prepared by conventional techniques known to those skilled in the art or by processes analogous to those described in the accompanying Examples include using an appropriate isotopically-labeled reagent in place of non-isotopically-labeled reagent.

Isotopically-enriched forms of compounds of formula (I), or any subformulas, may generally be prepared by conventional techniques known to those skilled in the art or by processes analogous to those described in the accompanying Examples using an appropriate isotopically-enriched reagent in place of a non-isotopically-enriched reagent. The extent of isotopic enrichment can be characterized as a percent incorporation of a particular isotope at an isotopically-labeled atom (e.g., % deuterium incorporation at a deuterium label).

### 3. Methods of Use

### Methods

In some embodiments, the compounds of formula (I) may decrease pro-inflammatory cytokines and/or increase anti-inflammatory cytokines. In another embodiment, methods of the present invention may comprise decreasing pro-inflammatory cytokines and/or increasing anti-inflammatory cytokines with a compound of formula (I) or a composition thereof. In another embodiment, methods of the present invention may be useful for treating or lessening the severity of a disease or disorder selected from a disease or disorder associated with an increase in pro-inflammatory cytokines comprising administering a therapeutically effective amount of the compounds of formula (I) or compositions thereof to a subject in need thereof.

In some embodiments, the compounds of formula (I) may promote oligodendrocyte proliferation, differentiation or survival. In one embodiment, the methods described herein also provide a method of promoting oligodendrocyte proliferation, differentiation or survival comprising contacting oligodendrocytes with a compound of formula (I) or a composition thereof.

In another embodiment, a method of the present invention comprises promoting oligodendrocyte proliferation, differentiation or survival. In another embodiment, a method of the present invention is useful for treating or lessening the severity of a disease or disorder selected from a disease or disorder associated with a lack of oligodendrocyte proliferation, differentiation or survival comprising administering a therapeutically effective amount of the compounds of formula (I) or compositions thereof to a subject in need thereof.

In some embodiments, the compounds of formula (I) may increase chemokines involved in oligodendrocyte precursor cell proliferation, differentiation and survival. In another embodiment, methods of the present invention may increase chemokines involved in oligodendrocyte precursor cell proliferation, differentiation and survival with a compound of formula (I) or a composition thereof.

In some embodiments, the compounds of formula (I) may suppress production of chemokines and/or cytokines which promote oligodendrocyte death. In another embodiment, methods of the present invention may decrease production of chemokines and/or cytokines involved in oligodendrocyte death with a compound of formula (I) or a composition thereof.

In another embodiment, a method of the present invention comprises promoting myelination by contacting neuronal cells, oligodendrocyte cells or oligodendrocyte precursor cells with a compound of formula (I) or a composition thereof.

In another embodiment, a method of the present invention is useful for treating or lessening the severity of a disease or disorder selected from a disease or condition associated with demyelination comprising administering a therapeutically effective amount of the compounds of formula (I) or compositions thereof to a subject in need thereof. In one embodiment, the disease or condition associated with demyelination is a CNS disorder or a CNS demyelinating disease as described herein. In one embodiment, the disease is multiple sclerosis.

In another embodiment, the subject has, or is at risk of having, multiple sclerosis. The subject with multiple sclerosis can be at any stage of treatment or disease. The subject with multiple sclerosis may have one or more of: benign multiple sclerosis, relapsing remitting multiple sclerosis, quiescent relapsing remitting multiple sclerosis, active relapsing remitting multiple sclerosis, progressive relapsing multiple sclerosis, primary progressive multiple sclerosis, or secondary progressive multiple sclerosis, clinically isolated syndrome, or clinically defined multiple sclerosis. The subject may be asymptomatic. The subject may have one or more multiple sclerosis-like symptoms, such as those having clinically isolated syndrome or clinically defined multiple sclerosis. The subject may have one or more multiple sclerosis relapses.

In some embodiments, the subject has a relapsing form of multiple sclerosis such as relapsing remitting multiple sclerosis or relapsing secondary progressive multiple sclerosis. In one embodiment, the subject has relapsing remitting multiple sclerosis and has one or more ongoing clinical exacerbations. In another embodiment, the subject has relapsing remitting multiple sclerosis and one or more subclinical activities. In one embodiment, the clinical exacerbation or subclinical activity may be shown by white matter lesions using magnetic resonance imaging.

In one embodiment, the clinical exacerbations or subclinical activities may be monitored by a functional readout such as ambulatory changes (gait changes, sway changes, etc.), T25W changes and or EDSS changes. In another embodiment, the clinical exacerbations or subclinical activities may be monitored by a visual evoked potential assay, a visual acuity assay, a measurement of optic nerve thickness or a myelin labelling assay.

The subject with multiple sclerosis can be at any stage of treatment or disease and treatment with compounds of formula (I) of the present invention result in improvement of the disease or symptoms. In one embodiment, improvement in the disease or symptoms is evidenced by a reduction or disappearance of one or more white matter lesions in the brain. In another embodiment, improvement in the disease or symptoms is evidenced by improved function such as improved ambulation, improved gait, reduced sway, improved T25W scores or improved EDSS scores. In another embodiment, improvement in the disease or symptoms is evidenced by improvements in a visual acuity assay or a visual evoked potential assay. In another embodiment, improvement in the disease or symptoms is evidenced by enhanced optic nerve thickness. In another embodiment, improvement in the disease or symptoms is evidenced by increased myelination in a myelin labelling assay.

In another embodiment, the compounds of formula (I) of the present invention and the methods, compositions and kits disclosed herein are useful for promoting myelin regeneration in progressive demyelinating diseases. In one embodiment, the compounds of formula I of the present invention and the methods, compositions and kits disclosed herein are useful for promoting myelin regeneration in primary progressive multiple sclerosis. In another embodiment, the compounds of formula I of the present invention and the methods, compositions and kits disclosed herein are useful for promoting myelin regeneration in secondary progressive multiple sclerosis. In another embodiment, the compounds of formula I of the present invention and the methods, compositions and kits disclosed herein are useful for promoting myelin regeneration in relapsing-remitting multiple sclerosis. In another embodiment, the compounds of formula I of the present invention and the methods, compositions and kits disclosed herein are useful for promoting myelin regeneration in progressive relapsing multiple sclerosis.

In yet another embodiment, the compounds of formula I of the present invention and the methods, compositions and kits disclosed herein are useful for promoting remyelination at the cellular level wherein oligodendrocyte cells are stimulated to regenerate or differentiate. In another embodiment, the compounds of formula I of the present invention and the methods, compositions and kits disclosed herein are useful for promoting remyelination at the cellular level wherein oligodendrocyte cells are stimulated to remyelinate axons.

In another embodiment, the compounds of formula I of the present invention and the methods, compositions and kits disclosed herein are useful for promoting remyelination at the cellular level whereby oligodendrocyte cells are stimulated to regenerate or differentiate thereby treating demyelinating diseases or disorders. In yet another embodiment, the compounds of formula I of the present invention and the methods, compositions and kits disclosed herein are useful for promoting remyelination at the cellular level whereby axons are remyelinated by oligodendrocyte cells thereby treating demyelinating diseases or disorders.

In another aspect, the present invention provides a method of treating or lessening the severity of, in a subject, a demyelinating disease comprising administering an effective amount of a compound, a pharmaceutically acceptable salt thereof or a pharmaceutical composition of the compounds of formula I. The demyelinating diseases may be a demyelinating myelinoclastic disease or a demyelinating leukodystrophic disease. The demyelinating myelinoclastic disease may be multiple sclerosis, Devic's disease or another inflammatory demyelinating disorder. The demyelinating leukodystrophic disease may be a central nervous system neuropathy, central pontine myelinolysis, a leukodystrophy, or another myelopathy. The demyelinating disease may affect the central nervous system or may affect the peripheral nervous system. Demyelinating disease of the the peripheral nervous system include: Guillain-Barré syndrome and its chronic counterpart, chronic inflammatory demyelinating polyneuropathy; anti-MAG peripheral neuropathy; Charcot-Marie-Tooth disease and its counterpart hereditary neuropathy with liability to pressure palsy; copper deficiency associated conditions (peripheral neuropathy, myelopathy, and rarely optic neuropathy); and progressive inflammatory neuropathy.

In another aspect, the present invention provides a method for treating, preventing or ameliorating one or more symptoms of multiple sclerosis or another neurodegenerative disease selected from auditory impairment, optic neuritis, decreased visual acuity, diplopia, nystagmus, ocular dysmetria, internuclear ophthalmoplegia, movement and sound phosphenes, afferent pupillary defect, paresis, monoparesis, paraparesis, hemiparesis, quadraparesis, plegia, paraplegia, hemiplegia, tetraplegia, quadraplegia, spasticity, dysarthria, motor dysfunction, walking impairment, muscle atrophy, spasms, cramps, hypotonia, clonus, myoclonus, myokymia, restless leg syndrome, gait disturbances, footdrop, dysfunctional reflexes, pallesthesia, anaesthesia, neuralgia, neuropathic and neurogenic pain, Lhermitte's, proprioceptive dysfunction, trigeminal neuralgia, ataxia, intention tremor, dysmetria, vestibular ataxia, vertigo, speech ataxia, dystonia, disability progression, dysdiadochokinesia, frequent micturition, bladder spasticity, flaccid bladder, detrusor-sphincter dyssynergia, erectile dysfunction or anorgasmy comprising administering an effective amount of a compound, a pharmaceutically acceptable salt thereof or a pharmaceutical composition of the compounds of formula I with one or more additional therapeutic agents administered concurrently with, prior to, or subsequent to treatment with the compound or pharmaceutical composition.

In another aspect, the present invention provides a method for treating, preventing or ameliorating one or more symptoms of endometriosis comprising administering an effective amount of a compound, a pharmaceutically acceptable salt thereof or a pharmaceutical composition of the compounds of formula I to a subject in need thereof.

### Administration

As described herein, compounds of the present invention, or a pharmaceutically acceptable salt thereof, or a pharmaceutical composition as described elsewhere herein, can be administered to such subjects by a variety of methods. In any of the uses or methods described herein, administration can be by various routes known to those skilled in the art, including without limitation oral, inhalation, intravenous, intramuscular, topical, subcutaneous, systemic, and/or intraperitoneal administration to a subject in need thereof.

The amount of the compound of the present invention, or a pharmaceutically acceptable salt thereof, required for use in treatment will vary not only with the particular compound or salt selected but also with the route of administration, the nature and/or symptoms of the estrogen receptor dependent and/or estrogen receptor mediated disease or condition being treated and the age and condition of the patient and will be ultimately at the discretion of the attendant physician or clinician. In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the dosage ranges described herein in order to effectively and aggressively treat particularly aggressive estrogen receptor dependent and/or estrogen receptor mediated diseases or conditions.

In some embodiments, the compounds, or pharmaceutically acceptable salts thereof, or pharmaceutical compositions as disclosed herein may be administered by inhalation, oral administration, or intravenous administration. In general, however, a suitable dose will often be in the range of from about 0.01 mg/kg to about 1000 mg/kg, such as from about 0.05 mg/kg to about 10 mg/kg. For example, a suitable dose may be in the range from about 0.10 mg/kg to about 10 mg/kg of body weight per day, such as about 0.10 mg/kg to about 0.50 mg/kg of body weight of the recipient per day, about 0.10 mg/kg to about 1.0 mg/kg of body weight of the recipient per day, about 0.15 mg/kg to about 5.0 mg/kg of body weight of the recipient per day, about 0.2 mg/kg to 4.0 mg/kg of body weight of the recipient per day. The compound may be administered in unit dosage form; for example, containing 1 to 100 mg, 10 to 100 mg, or 5 to 50 mg of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

As will be readily apparent to one skilled in the art, the useful in vivo dosage to be administered and the particular mode of administration will vary depending upon the age, weight, the severity of the affliction, and mammalian species treated, the particular compounds employed, and the specific use for which these compounds are employed. The determination of effective dosage levels, that is the dosage levels necessary to achieve the desired result, can be accomplished by one skilled in the art using routine methods, for example, human clinical trials, in vivo studies and in vitro studies. For example, useful dosages of a compound of the present invention, or pharmaceutically acceptable salts thereof, can be determined by comparing their in vitro activity, and in vivo activity in animal models. Such comparison can be done by comparison against an established drug, such as fulvestrant.

Dosage amount and interval may be adjusted individually to provide plasma levels of the active moiety which are sufficient to maintain the modulating effects, or minimal effective concentration (MEC). The MEC will vary for each compound but can be estimated from in vivo and/or in vitro data. Dosages necessary to achieve the MEC will depend on individual characteristics and route of administration. However, FIPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the estrogen receptor dependent and/or estrogen receptor mediated disease or condition to be treated and to the route of administration. The severity of the estrogen receptor dependent and/or estrogen receptor mediated disease or condition may, for example, be evaluated, in part, by standard prognostic evaluation methods. Further, the dose, and perhaps dose frequency, will also vary according to the age, body weight, and response of the individual patient. A program comparable to that discussed above may be used in veterinary medicine.

Compounds, salts and compositions disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

A therapeutically effective amount of a compound disclosed herein, or a pharmaceutically acceptable salt thereof, or a pharmaceutical composition as disclosed herein may be administered alone or in combination with a therapeutically effective amount of at least one additional therapeutic agent. In some embodiments, the compounds or pharmaceutical compositions as disclosed herein are administered in combination with at least one additional therapeutic agent. In some embodiments, the at least one additional therapeutic is administered prior to or following administration of the compounds or pharmaceutical compositions as disclosed herein. In some embodiments, compounds and compositions of the invention may be administered in combination with one or more of interferon beta-la, interferon beta-1b, glatiramer acetate, peginterferon beta-la, daclizumab, teriflunomide, fingolimod, dimethyl fumarate, alemtuzumab, mitoxantrone, ocrelizumab, or natalizumab. In some embodiments, compounds and compositions of the invention may be administered in combination with one or more of methylprednisolone, prednisone, ACTH, onabotulinumtoxin A, desmopressin, tolterodine, oxybutynin, darifenacin, tamsulosin, terazosin, prazosin, mirabegron, propantheline, trospium chloride, imipramine, solifenacin succinate, dantrolene, baclofen, clonazepam, diazepam, tizanidine, isoniazid, clonazepam, or dalfampridine.

### 4. Pharmaceutical Compositions

In another aspect of the invention, pharmaceutically acceptable compositions are provided, wherein these compositions comprise any of the compounds as described herein, and optionally comprise a pharmaceutically acceptable carrier, adjuvant or vehicle. In certain embodiments, these compositions optionally further comprise one or more additional therapeutic agents. In one embodiment, the pharmaceutical composition comprises a therapeutically effective amount of a compound of the present invention or a pharmaceutically acceptable salt thereof and one or more pharmaceutically acceptable carriers or vehicles.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

As used herein, the term “pharmaceutically acceptable salt” refers to those salts which are, within the scope of sound medical judgement, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N(C1-4 alkyl)₄ salts. This invention also envisions the quaternization of any basic nitrogen-containing groups of the compounds disclosed herein. Water or oil-soluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl (e.g., phenyl/substituted phenyl) sulfonate.

As described herein, the pharmaceutically acceptable compositions of the invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylenepolyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutically acceptable compositions of this invention can be administered to humans and other animals orally, rectally, parenterally, intracistemally, intravaginally, intraperitoneally, topically (as by powders, ointments, or drops), buccally, as an oral or nasal spray, or the like, depending on the severity of the disease being treated.

Pharmaceutical compositions for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), vegetable oils (such as olive oil), injectable organic esters (such as ethyl oleate) and suitable mixtures thereof. Proper fluidity can be maintained, for example, by the use of coating materials such as lecithin, by the maintenance of the required particle size in the case of dispersions and by the use of surfactants.

These compositions can also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid and the like. It can also be desirable to include isotonic agents such as sugars, sodium chloride and the like. Prolonged absorption of the injectable pharmaceutical form can be brought about by the inclusion of agents which delay absorption such as aluminum monostearate and gelatin.

In some cases, in order to prolong the effect of the drug, it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, can depend upon crystal size and crystalline form. Alternatively, delayed absorption of a parenterally administered drug form is accomplished by dissolving or suspending the drug in an oil vehicle.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, cement, putty, and granules. In such solid dosage forms, the active compound can be mixed with at least one inert, pharmaceutically acceptable excipient or carrier, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form can also comprise buffering agents.

Solid compositions of a similar type may also be employed as fillers in soft and hard filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Dosage forms for topical or trans dermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the invention contemplates the use of transdermal patches, which have the added advantage of providing controlled delivery of a compound to the body. Such dosage forms are prepared by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Compounds described herein can be administered as a pharmaceutical composition comprising the compounds of interest in combination with one or more pharmaceutically acceptable carriers. It is understood, however, that the total daily dosage of the compounds and compositions can be decided by the attending physician within the scope of sound medical judgment. The specific therapeutically effective dose level for any particular patient can depend upon a variety of factors including the disorder being treated and the severity of the disorder; activity of the specific compound employed; the specific composition employed; the age, body weight, general health and prior medical history, sex and diet of the patient; the time of administration, route of administration, and rate of excretion of the specific compound employed; the duration of the treatment; drugs used in combination or coincidental with the specific compound employed; and like factors well-known in the medical arts. For example, it is well within the skill of the art to start doses of the compound at levels lower than required to achieve the desired therapeutic effect and to gradually increase the dosage until the desired effect is achieved. Actual dosage levels of active ingredients in the pharmaceutical compositions can be varied so as to obtain an amount of the active compound(s) that is effective to achieve the desired therapeutic response for a particular patient and a particular mode of administration. In the treatment of certain medical conditions, repeated or chronic administration of compounds can be required to achieve the desired therapeutic response. “Repeated or chronic administration” refers to the administration of compounds daily (i.e., every day) or intermittently (i.e., not every day) over a period of days, weeks, months, or longer.

The compositions described herein may be administered with additional compositions to prolong stability, delivery, and/or activity of the compositions, or combined with additional therapeutic agents, or provided before or after the administration of additional therapeutic agents.

Combination therapy includes administration of a single pharmaceutical dosage formulation containing one or more of the compounds described herein and one or more additional pharmaceutical agents, as well as administration of the compounds and each additional pharmaceutical agent, in its own separate pharmaceutical dosage formulation. For example, a compound described herein and one or more additional pharmaceutical agents, can be administered to the patient together, in a single oral dosage composition having a fixed ratio of each active ingredient, such as a tablet or capsule; or each agent can be administered in separate oral dosage formulations. Where separate dosage formulations are used, the present compounds and one or more additional pharmaceutical agents can be administered at essentially the same time (e.g., concurrently) or at separately staggered times (e.g., sequentially).

For adults, the doses are generally from about 0.01 to about 100 mg/kg, desirably about 0.1 to about 1 mg/kg body weight per day by inhalation, from about 0.01 to about 100 mg/kg, desirably 0.1 to 70 mg/kg, more desirably 0.5 to 10 mg/kg body weight per day by oral administration, and from about 0.01 to about 50 mg/kg, desirably 0.1 to 1 mg/kg body weight per day by intravenous administration.

The compositions and methods will be described more fully hereinafter with reference to the accompanying drawings, in which some, but not all embodiments of the invention are shown. Indeed, the invention may be embodied in many different forms and should not be construed as limited to the embodiments set forth herein.

Likewise, many modifications and other embodiments of the compositions and methods described herein will come to mind to one of skill in the art to which the invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

Unless defined otherwise, all technical and scientific terms used herein have the same meaning as commonly understood by one of skill in the art to which the invention pertains. Although any methods and materials similar to or equivalent to those described herein can be used in the practice or testing of the present invention, the preferred methods and materials are described herein.

### 5. Chemical Synthesis

Compounds of the invention may be prepared as illustrated in the following schemes and examples.

### Abbreviations

Ac acetate
Ac₂O acetic anhydride
aq. aqueous
Calcd calculated
cat. catalytic
c-HCl concentrated HCl
DCM dichloromethane
DI deionized
DMF N,N-dimethylformamide
eq. equivalent(s)
Et ethyl
ESI electrospray ionization
EtOAc ethyl acetate
h or hr hour
HRMS high resolution mass spectrometry
Me methyl
MeOH methanol
min. minute(s)
NBS N-bromosuccinimide
NCI negative chemical ionization
NCS N-chlorosuccinimide
NIS N-iodosuccinimide
Ph phenyl
ppt precipitate
pTsOH/TsOH p-toluenesulfonic acid
pyr pyridine
rt or r.t. room temperature
sat. saturated
Selectfluor® 1-Chloromethyl-4-fluoro-1,4-diazoniabicyclo[2.2.2]octane bis(tetrafluoroborate)
TBAF tetrabutylammonium fluoride
THF tetrahydrofuran
TLC thin layer chromatography

Scheme 1 shows a general method for synthesis of compounds of formula (I), wherein R⁵ is substituted in the para position. Compounds wherein R⁵ is substituted in the meta position may be synthesized by similar processes starting from appropriate meta substituted anilines.

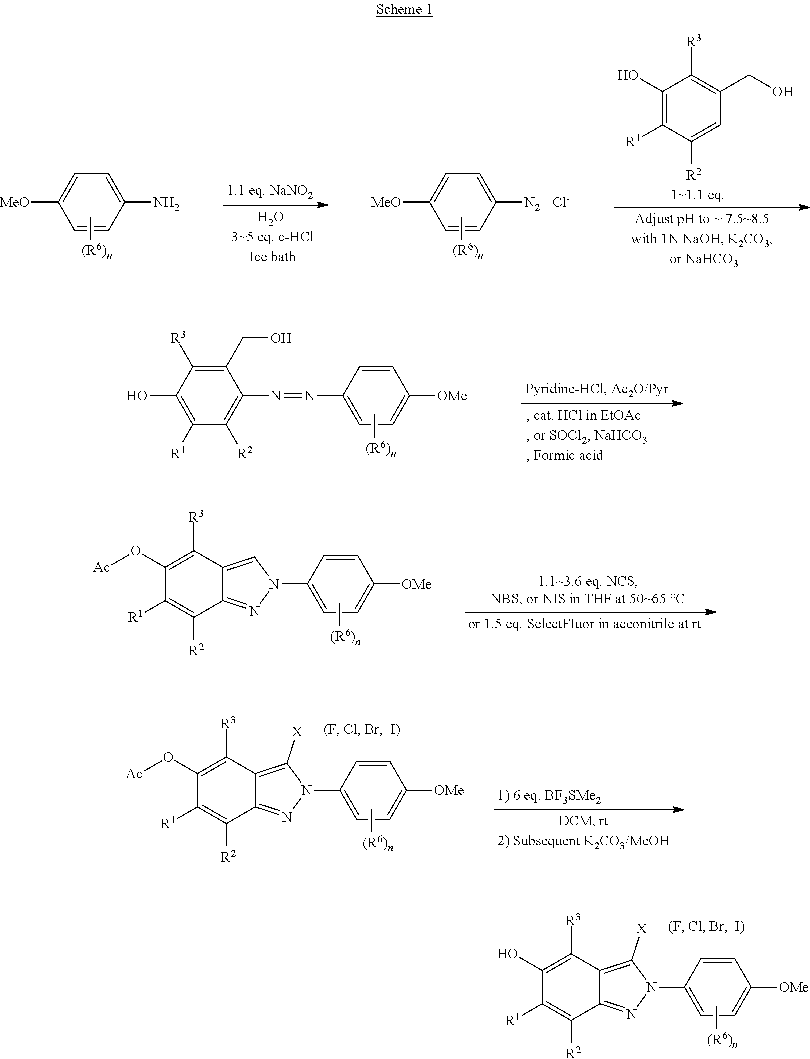

### Chemical Examples

### 1). Synthesis of 7-bromo-2-(4-hydroxyphenyl)-2H-indazol-5-ol (Ind-7-Br, 1)

### 1-1) Synthesis of 7-bromo-2-(4-methoxyphenyl)-2H-indazol-5-yl acetate (5-Ac-Ind-7-Br-4′-OMe, 1-1)

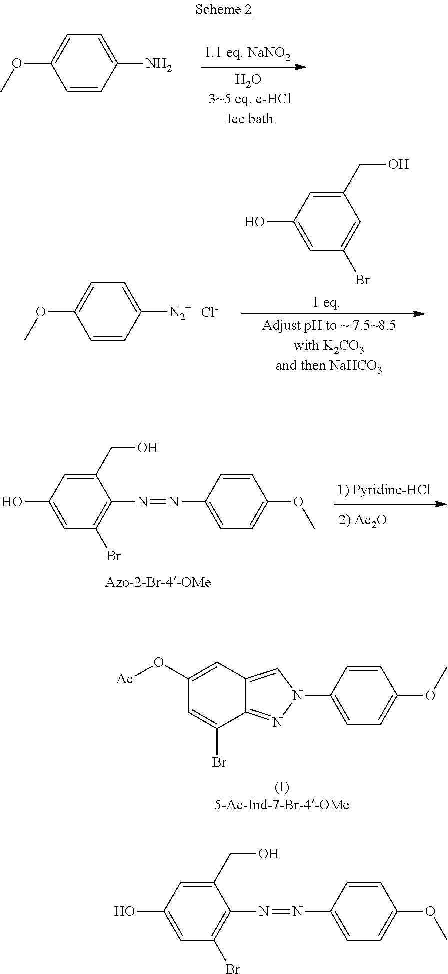

3-Bromo-5-(hydroxymethyl)-4-((4-methoxyphenyl)diazenyl)phenol (AZO-2-Br-4′-OMe). To the suspension of 4-methoxyaniline (123 mg, 1.00 mmol) in cold DI water (10 mL) was added c-HCl (0.36 mL) and subsequently, sodium nitrite (71 mg, 1.03 mmol) was added portion-wise in an ice bath by maintaining the temperature below 5° C. After 1.5 hr stirring of the reaction mixture at below 5° C., 3-bromo-5-hydroxymethyl phenol (190 mg, 0.94 mmol) in a water-ethanol (1:2, v/v, 5 ml) mixture was added to the reaction mixture in an ice bath, followed by adjusting the pH up to 7.5 with K₂CO₃ cautiously to precipitate a dark brown solid. During the addition of the K₂CO₃, a massively brown solid precipitated from the solution as the pH reached ~7.5. The solid was collected by filtration, washed with DI water, and dried in an oven at 60~80° C., affording a brown solid AZO-2-Br-4′-OMe (253 mg). (It is not necessary to adjust pH acidic in this case to collect a solid, but in some cases, the pH of the solution needs to be acidified to collect more solid.). The collected solid was used without further purification. ¹H NMR (499 MHz, CD₃OD) δ 7.86 (d, J=8.5 Hz, 2H), 7.17 (d, J=2.7 Hz, 1H), 7.01 (d, J=8.5 Hz, 2H), 6.97 (d, J=2.7 Hz, 1H), 4.53 (s, 2H), 3.88 (s, 3H). ESI (m/z, M+1) 337.02, 339.02.

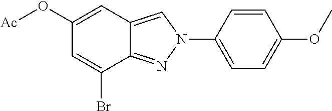 [SMILES: COc1ccc(-n2cc3cc(OC(C)=O)cc(Br)c3n2)cc1]

5-Ac-Ind-7-Br-4′-OMe (1-1). Pyridine HCl salt (500 mg) was placed in a 50 mL round bottom flask and was heated up at 165° C. in the oil bath until it fully melted. The dried brown solid AZO-2-Br-4′-OMe (152 mg, 0.45 mmol) was added to the melted pyridine HCl solution and heated in the mixture for 5 min. Once all the starting material is converted to the compound indazole as monitored by SiO₂ TLC, on which the indazole at 254 nm UV exposure shows a weak blue fluorescence, acetic anhydride (300 μL) and pyridine (100 μL) were added to the melted reaction mixture and water (50 mL) was added to the reaction solution resulting in formation of a grey precipitate. The precipitate was collected by filtration and dried under vacuum to afford compound 1-1 as a grey solid (140 mg). The collected and dried compound (1-1) is pure enough to use without further purification. ¹H NMR (499 MHz, CDCl₃) δ 8.38 (s, 1H), 7.81 (d, J=10.05 Hz, 2H), 7.40 (d, J=2.0 Hz, 1H), 7.32 (d, J=2.0 Hz, 1H), 7.03 (d, J=10.05 Hz, 2H), 3.88 (s, 3H), 2.32 (s, 3H). ¹³C NMR (126 MHz, CDCl₃) δ 170.77, 159.45, 146.63, 144.94, 135.86, 126.34, 123.65, 122.39, 122.22, 116.23, 112.86, 110.64, 53.72, 19.43.ESI (m/z, M+1) 361.02, 363.02.

### 1-2) Synthesis of 7-bromo-2-(4-hydroxyphenyl)-2H-indazol-5-ol (Ind-7-Br, 1)

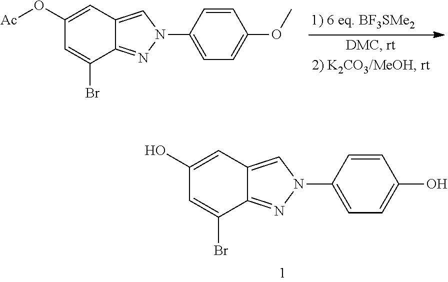 [SMILES: COc1ccc(-n2cc3cc(OC(C)=O)cc(Br)c3n2)cc1 ; Oc1ccc(-n2cc3cc(O)cc(Br)c3n2)cc1]

7-Bromo-2-(4-methoxyphenyl)-2H-indazol-5-yl acetate (37.5 mg, 0.10 mmol) was dissolved into dichloromethane (5 mL). To the resulting solution, boron trifluoride dimethyl sulfide (200 mg, 1.54 mmol) was added, and the reaction vessel was stirred for 6 hr at rt. Once the starting material had disappeared upon silica gel TLC analysis, the solvent and excess of the boron trifluoride dimethyl sulfide was evaporated. To the resultant product was added potassium carbonate (25 mg) and methanol (500 μL) and the mixture was stirred for 1 hr at rt, solvent evaporated and treated with 1 N HCl water (100 μL) to afford a precipitate, which was collected by filtration to provide a pale brownish solid, which was dried to afford the title compound (Ind-7-Br) (26 mg). ¹HNMR (500 MHz, CD₃OD-CDCl₃) δ 8.26 (s, 1H), 7.64 (d, J=8.9 Hz, 2H), 7.23 (d, J=2.1 Hz, 1H), 6.93 (d, J=8.9 Hz, 2H), 6.92 (d, J=2.1 Hz, 1H). ¹³C NMR (126 MHz, CD₃OD-CDCl₃) δ 157.05, 153.10, 145.93, 134.10, 124.53, 123.57, 123.17, 121.63, 118.02, 109.26, 102.24. ESI (m/z, M+1) 304.99, 306.99.

### 2). Synthesis of 7-bromo-3-fluoro-2-(4-hydroxyphenyl)-2H-indazol-5-ol (IndF-7-Br, 2)

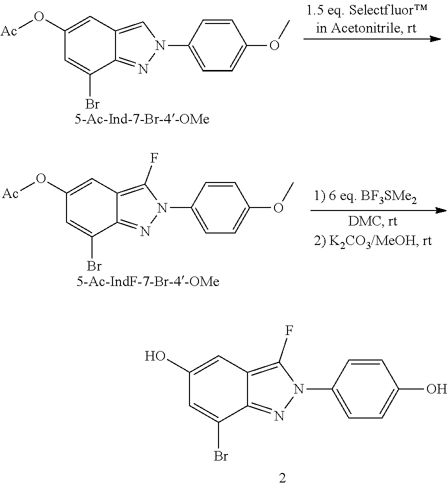 [SMILES: COc1ccc(-n2cc3cc(OC(C)=O)cc(Br)c3n2)cc1 ; COc1ccc(-n2nc3c(Br)cc(OC(C)=O)cc3c2F)cc1 ; Oc1ccc(-n2nc3c(Br)cc(O)cc3c2F)cc1]

5-Ac-IndF-7-Br-4′-OMe was prepared using a fluorination procedure described in WO2019226936. 5-Ac-Ind-7-Br-4′-OMe (36.1 mg, 0.10 mmol) was treated with Selectfluor™ (1-chloromethyl-4-fluoro-1,4-diazoniabicyclo[2.2.2]octane bis(tetrafluoroborate)) (49.28 mg, 0.14 mmol) in acetonitrile (3 mL) for 8 hr at rt until 5-Ac-Ind-7-Br-4′-OMe disappeared on silica gel thin layer chromatography (20% EtOAc in n-Hexane, Rf=0.5). The reaction mixture was poured into 20 mL of water, extracted with 10 mL of EtOAc three times, dried using sodium sulfate, and then the EtOAc layer was concentrated and loaded onto silica gel for column chromatography. Elution with a mixture of ethyl acetate and n-hexane (1:5=v/v) afforded 5-Ac-IndF-7-Br-4′-OMe in 69% yield. Subsequent treatment of 5-Ac-IndF-7-Br-4′-OMe with BF₃—SMe₂ (20 eq.) in DCM and K₂CO₃ in MeOH afforded IndF-7-Br (2) in 73% yield, following the same method as described to make IndBr-7-Br. ¹H NMR (499 MHz, CDCl₃) δ 7.53 (dd, J=8.8, 1.7 Hz, 2H), 7.26 (d, J=2.0 Hz, 1H), 6.93 (d, J=8.9 Hz, 2H), 6.75 (d, J=2.0 Hz, 1H). ¹⁹F NMR (470 MHz, CDCl₃) δ −133.53. ESI (m/z, M+1) 323.0, 325.0.

### 3). Synthesis of 7-bromo-3-chloro-2-(4-hydroxyphenyl)-2H-indazol-5-ol (IndCl-7-Br, 3)

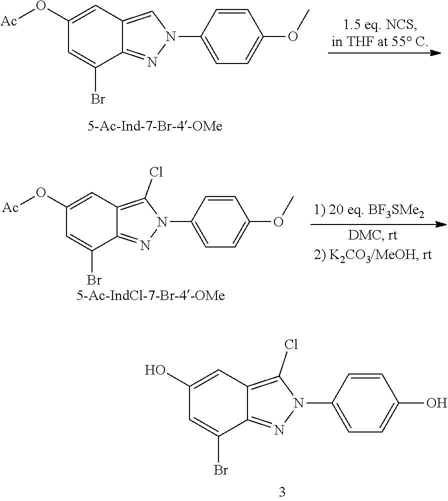 [SMILES: COc1ccc(-n2cc3cc(OC(C)=O)cc(Br)c3n2)cc1 ; COc1ccc(-n2nc3c(Br)cc(OC(C)=O)cc3c2Cl)cc1 ; Oc1ccc(-n2nc3c(Br)cc(O)cc3c2Cl)cc1]

IndCl-7-Br (3) was obtained in 77% yield from the reaction of 5-Ac-Ind-7-Br-4′-OMe (36.1 mg, 0.10 mmol) with NCS (20 mg, 0.15 mmol) and a catalytic amount of p-TsOH using the same method to make 5-Ac-IndBr-7-Br-4′-OMe, and subsequent treatment of 5-Ac-IndCl-7-Br-4′-OMe with BF₃—SMe₂ (20 eq.) in DCM at rt for 6 hr and then K₂CO₃ by following the method to make IndBr-7-Br. ¹H NMR (499 MHz, CD₃OD) δ 7.39 (d, J=8.9 Hz, 2H), 7.27 (d, J=2.1 Hz, 1H), 6.94 (d, J=8.9 Hz, 2H), 6.77 (d, J=2.1 Hz, 1H). ESI (m/z, M+1) 338.9, 340.9, 342.9.

### 4). Synthesis of 3,7-dibromo-2-(4-hydroxyphenyl)-2H-indazol-5-ol (IndBr-7-Br, 4)

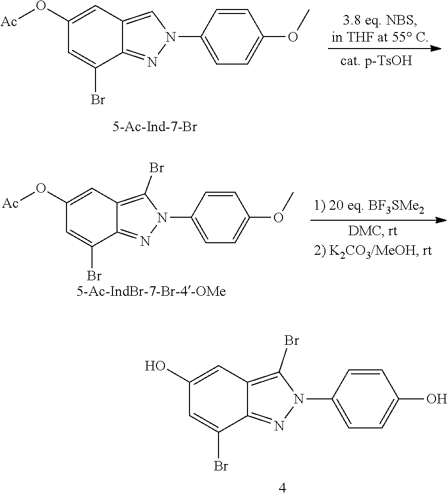 [SMILES: COc1ccc(-n2cc3cc(OC(C)=O)cc(Br)c3n2)cc1 ; COc1ccc(-n2nc3c(Br)cc(OC(C)=O)cc3c2Br)cc1 ; Oc1ccc(-n2nc3c(Br)cc(O)cc3c2Br)cc1]

3,7-Dibromo-2-(4-methoxyphenyl)-2H-indazol-5-yl acetate (5-Ac-IndBr-7-Br-4′-OMe). To the acetyl compound (5-Ac-Ind-7-Br) (75.0 mg, 0.200 mmol) in THF (5 mL) was added NBS (N-bromosuccinimide) (128.0 mg, 0.76 mmol) and cat. amount of p-TsOH, and the resulting solution was stirred for 4 hr at 55° C. while monitoring the reaction by silica gel TLC. The resulting solution was concentrated and loaded on silica gel coated preparative TLC (thickness 1 mm, Plate L×W=20 cm×20 cm). The development with the mixture of ethyl acetate and n-hexane (3:7=v/v) afforded 5-Ac-IndBr-7-Br-4′-OMe (70 mg) as a pale brownish solid. ¹H NMR (499 MHz, CDCl₃) δ 7.59 (d, J=8.9 Hz, 2H), 7.39 (d, J=2.0 Hz, 1H), 7.32 (d, J=2.0 Hz, 1H), 7.07 (d, J=8.9 Hz, 2H), 3.91 (s, 3H), 2.35 (s, 3H). ESI (m/z, M+1) 438.9, 440.9, 442.9.

3,7-Dibromo-2-(4-hydroxyphenyl)-2H-indazol-5-ol (IndBr-7-Br, 4). The solid 5-Ac-IndBr-7-Br-4′-OMe was dissolved into dichloromethane (5 mL) and treated with boron trifluoride dimethyl sulfide (520 mg, 4.00 mmol) at rt. Once the starting material disappeared upon silica gel TLC, the solvent and excess boron trifluoride dimethyl sulfide were evaporated. Subsequently, K₂CO₃ (50 mg, 0.36 mmol) and MeOH (1.0 mL) were added to the residue, followed by sonication for 10 min., evaporation of solvent MeOH, acidification with 0.1 N HCl solution, extraction with ethyl acetate (1 mL×3), and evaporation to afford the title IndBr-7-Br (58 mg) (4) as a pale brownish solid. ¹H NMR (499 MHz, CD₃OD) δ 7.37 (d, J=8.4 Hz, 2H), 7.28 (d, J=2.1 Hz, 1H), 6.91 (d, J=8.9 Hz, 2H), 6.73 (d, J=2.1 Hz, 1H). ESI (m/z, M+1) 382.9 (50%), 384.9 (100%), 386.9 (49%).

### 5). Synthesis of 7-bromo-2-(4-hydroxyphenyl)-3-iodo-2H-indazol-5-ol (IndI-7-Br, 5)

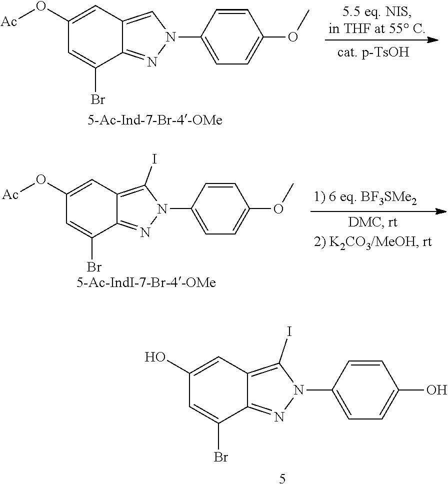 [SMILES: COc1ccc(-n2cc3cc(OC(C)=O)cc(Br)c3n2)cc1 ; COc1ccc(-n2nc3c(Br)cc(OC(C)=O)cc3c2I)cc1 ; Oc1ccc(-n2nc3c(Br)cc(O)cc3c2I)cc1]

5-Ac-IndI-7-Br-4′-OMe. Reaction of 5-Ac-Ind-7-Br-4′-OMe (36.1 mg, 0.10 mmol) with NIS (123.74 mg, 0.55 mmol) and a catalytic amount of p-TsOH using the same method to make 5-Ac-IndBr-7-Br-4′-OMe provided 5-Ac-IndI-7-Br-4′-OMe. ¹H NMR (499 MHz, CDCl₃) δ 7.54 (d, J=9.1 Hz, 2H), 7.40 (d, J=2.1 Hz, 1H), 7.23 (d, J=2.1 Hz, 1H), 7.06 (d, J=9.1 Hz, 2H), 3.92 (s, 3H), 2.36 (s, 3H). ¹³C NMR (126 MHz, CDCl₃) δ 168.80, 162.61, 150.13, 146.21, 133.53, 130.21, 127.74, 127.02, 117.38, 113.16, 111.85, 52.71, 20.91. ESI (m/z, M+1) 486.9, 488.9.

IndI-7-Br (5). The title compound was obtained by reaction of 5-Ac-IndI-7-Br-4′-OMe with BF₃—SMe₂ (20 eq.) in DCM at rt for 6 hr, and subsequent treatment with potassium carbonate by following the method to make IndBr-7-Br. ¹H NMR (499 MHz, CD₃OD) δ 7.33 (d, J=8.8 Hz, 2H), 7.29 (d, J=2.1 Hz, 1H), 6.92 (d, J=8.8 Hz, 2H), 6.66 (d, J=2.1 Hz, 1H). ESI (m/z, M+1) 430.9, 432.9.

### 6). Synthesis of 7-chloro-2-(4-hydroxyphenyl)-2H-indazol-5-ol (Ind-7-Cl, 6)

### 6-1). Chloro-5-(hydroxymethyl)-4-((4-methoxyphenyl)diazenyl)phenol (AZO-2-Cl-4′-OMe)

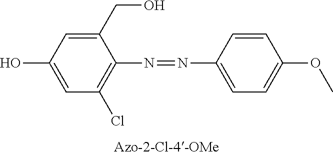 [SMILES: COc1ccc(N=Nc2c(Cl)cc(O)cc2CO)cc1]

AZO-2-Cl-4′-OMe was obtained from the reaction of p-methoxyaniline (123.0 mg, 1.00 mmol) with NaNO₂ (72.0 mg, 1.03 mmol) and 3-chloro-5-hydroxymethyl phenol (145.0 mg, 0.91 mmol) in 85% yield as yellowish solid, following the same method as described to make Azo-2-Br-4′-OMe compound as shown in scheme 2. ¹H NMR (499 MHz, CD₃OD) δ 7.85 (d, J=9.0 Hz, 2H), 7.00 (d, J=9.0 Hz, 2H), 6.98 (d, J=2.7 Hz, 1H), 6.83 (d, J=2.7 Hz, 1H), 4.43 (s, 2H), 3.87 (s, 3H). ESI (m/z, M+1) 293 (100%), 295 (31%).

### 6-2). 5-Ac-Ind-7-Cl-4′-OMe (II)

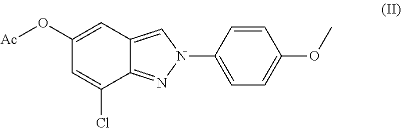 [SMILES: COc1ccc(-n2cc3cc(OC(C)=O)cc(Cl)c3n2)cc1]

5-Ac-Ind-7-Cl-4′-OMe (II) was obtained from the treatment of AZO-2-Cl-4′-OMe (292.0 mg, 1.00 mmol) with pyridine HCl (700.0 mg) and acetic anhydride (1 mL) in 78%, following the same method as described to make 5-Ac-Ind-7-Br-4′-OMe. ¹H NMR (499 MHz, CDCl₃) δ 8.35 (s, 1H), 7.79 (d, J=9.0 Hz, 2H), 7.34 (d, J=2.0 Hz, 1H), 7.14 (d, J=2.0 Hz, 1H), 7.03 (d, J=9.0 Hz, 2H), 3.87 (s, 3H), 2.33 (s, 3H). ¹³C NMR (126 MHz, CDCl₃) δ 169.88, 159.98, 145.68, 145.43, 133.92, 124.11, 123.06, 122.81, 122.35, 122.30, 114.93, 110.20, 55.91, 21.35. ESI (m/z, M+1) 317.0 (100%), 319 (31%).

### 6-3). 7-Chloro-2-(4-hydroxyphenyl)-2H-indazol-5-ol (Ind-7-Cl, 6)

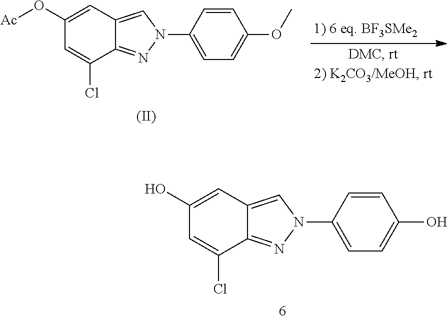

Ind-7-Cl (6) was prepared from the treatment of 5-Ac-Ind-7-Cl-4′-MeO (II) (31.7 mg, 0.10 mmol) with BF₃—SMe₂ (20 eq.) in DCM and then with K₂CO₃ in MeOH in 91% yield, following the same method as described to make Ind-7-Br (1). ¹H NMR (499 MHz, CD₃OD) δ 8.16 (s, 1H), 7.60 (d, J=8.9 Hz, 2H), 7.03 (d, J=2.0 Hz, 1H), 6.91 (d, J=8.9 Hz, 2H), 6.85 (d, J=2.0 Hz, 1H). ¹³C NMR (126 MHz, Methanol-d₄) δ 157.56, 152.13, 143.73, 132.95, 123.96, 123.18, 123.00, 121.42, 120.48, 116.15, 99.42. ESI (m/z, M+1) 261.03 (100%), 263.02 (30%).

### 7). Synthesis of 3,7-dichloro-2-(4-hydroxyphenyl)-2H-indazol-5-ol (IndCl-7-Cl, 7)

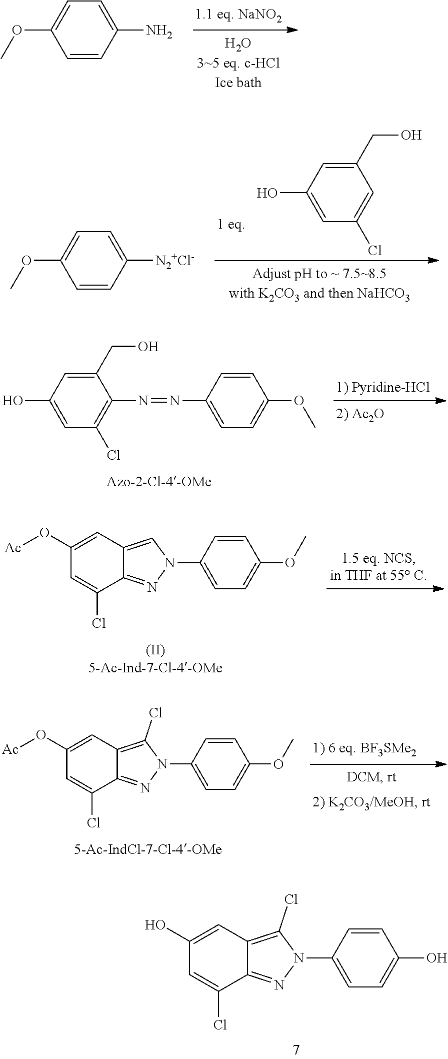

5-Ac-IndCl-7-Cl-4′-OMe. Reaction of 5-Ac-Ind-7-Cl-4′-OMe (II) (31.7 mg, 0.10 mmol) with NCS (20.0 mg, 0.15 mmol) provided 5-Ac-IndCl-7-Cl-4′-OMe. ¹H NMR (499 MHz, CDCl₃) δ 7.57 (d, J=9.0 Hz, 2H), 7.28 (d, J=2.0 Hz, 1H), 7.16 (d, J=2.0 Hz, 1H), 7.03 (d, J=9.0 Hz, 2H), 3.87 (s, 3H), 2.33 (s, 3H). ¹³C NMR (126 MHz, CDCl₃) δ 169.75, 160.66, 145.71, 144.34, 131.27, 127.46, 124.46, 123.39, 121.67, 119.93, 114.59, 109.14, 55.92, 21.30.

IndCl-7-Cl (7). The title compound was obtained by reaction of 5-Ac-IndCl-7-Cl-4′-OMe with BF₃SMe₂ (12 eq.) in DCM and K₂CO₃ in MeOH, following the same method as described to make IndBr-7-Br. ¹H NMR (499 MHz, CD₃OD) δ 7.39 (d, J=8.8 Hz, 2H), 7.07 (d, J=2.0 Hz, 1H), 6.93 (d, J=8.8 Hz, 2H), 6.72 (d, J=2.1 Hz, 1H). ¹³C NMR (126 MHz, CD₃OD) δ 157.83, 153.69, 143.65, 131.19, 127.47, 123.48, 121.69, 120.84, 119.22, 116.88, 109.45. ESI (m/z, M+1) 295.0 (100%), 297 (61%).

### 8). Synthesis of 7-fluoro-2-(4-hydroxyphenyl)-2H-indazol-5-ol (Ind-7-F, 8)

### 3-Fluoro-5-(hydroxymethyl)-4-((4-methoxyphenyl)diazenyl)phenol (AZO-2-F-4′-OMe)

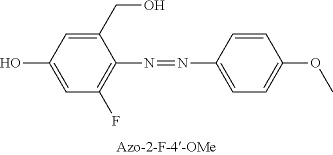 [SMILES: COc1ccc(N=Nc2c(F)cc(O)cc2CO)cc1]

AZO-2-F-4′-OMe was obtained from the reaction of p-methoxyaniline (123.0 mg, 1.00 mmol) with NaNO₂ (72.0 mg, 1.03 mmol) and 3-fluoro-5-hydroxymethyl phenol (142.0 mg, 1.00 mmol) in 93% yield as yellowish solid, following the same method as described to make Azo-2-Br-4′-OMe compound. ¹H NMR (499 MHz, CD₃OD) δ 7.81 (d, J=9.0 Hz, 1H), 6.99 (d, J=9.0 Hz, 1H), 6.78 (d, J=2.7 Hz, 1H), 6.62 (dd, J=12.6, 2.7 Hz, 1H), 4.64 (s, 2H), 3.86 (s, 3H). ¹⁹F NMR (470 MHz, CD₃OD) δ −119.22 (d, J=12.9 Hz).

### 5-Ac-Ind-7-F-4′-OMe (III)

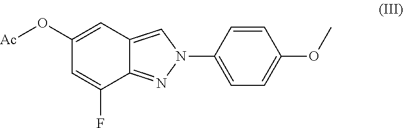 [SMILES: COc1ccc(-n2cc3cc(OC(C)=O)cc(F)c3n2)cc1]

5-Ac-Ind-7-F-4′-OMe (III) was obtained from the treatment of AZO-2-F-4′-OMe (292.0 mg, 1.00 mmol) with pyridine HCl (700.0 mg) and acetic anhydride (1 mL) in 91% yield, following the same method as described to make 5-Ac-Ind-7-Br-4′-OMe. ¹H NMR (499 MHz, CDCl₃) δ 8.33 (d, J=2.6 Hz, 1H), 7.78 (d, J=9.0 Hz, 2H), 7.21 (d, J=1.8 Hz, 1H), 7.01 (d, J=9.0 Hz, 2H), 6.76 (dd, J=11.1, 1.8 Hz, 1H), 3.86 (s, 3H), 2.31 (s, 3H). ¹⁹F NMR (470 MHz, CDCl₃) δ −125.74 (d, J=11.4 Hz).

### 7-Fluoro-2-(4-hydroxyphenyl)-2H-indazol-5-ol (Ind-7-F, 8)

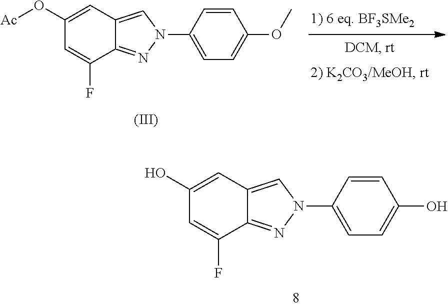

Ind-7-F (8) was prepared from the treatment of 5-Ac-Ind-7-F-4′-OMe (III) (30.0 mg, 0.10 mmol) with BF₃—SMe₂ (20 eq.) and K₂CO₃ in 85% yield, following the same method as described to make Ind-7-Br (1). ¹H NMR (499 MHz, CD₃OD) δ 8.23 (s, 1H), 7.61 (d, J=8.88 Hz, 2H), 6.91 (d, J=8.88 Hz, 2H), 6.73 (d, J=1.85 Hz, 1H), 6.67 (dd, J=12.33, 1.85 Hz, 1H). ¹⁹F NMR (470 MHz, CD₃OD) δ 128.75 (d, J=12.38 Hz). ESI (m/z, M+1) 245.07 (100%).

### 9). Synthesis of 3-Chloro-7-fluoro-2-(4-hydroxyphenyl)-2H-indazol-5-ol (IndCl-7-F, 9)

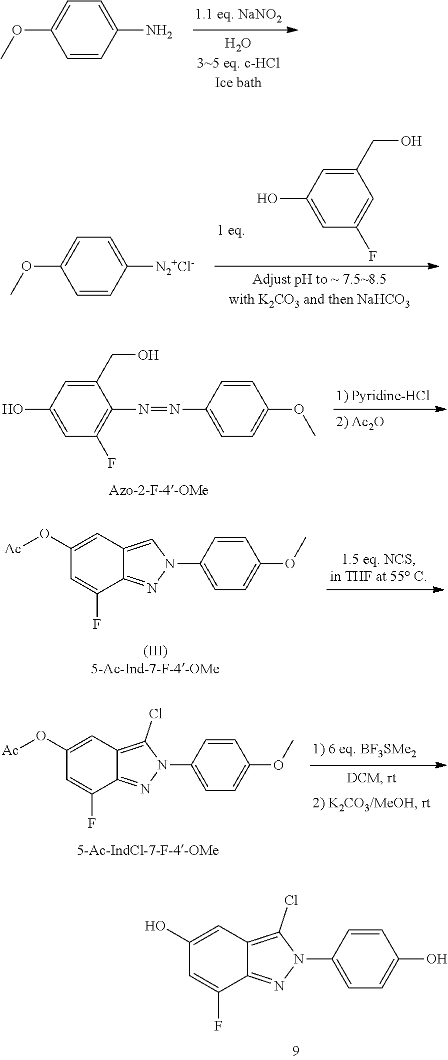

5-Ac-IndCl-7-F-4′-OMe was obtained by reaction of 5-Ac-Ind-7-F-4′-OMe (III) (30.0 mg, 0.10 mmol) with NCS (20.0 mg, 0.15 mmol). ¹H NMR (499 MHz, CDCl₃) δ 7.58 (d, J=9.0 Hz, 1H), 7.16 (d, J=1.8 Hz, 1H), 7.04 (d, J=9.0 Hz, 1H), 6.80 (dd, J=10.9, 1.9 Hz, 1H), 3.88 (s, 2H), 2.33 (s, 2H). ¹⁹F NMR (470 MHz, CDCl₃) δ −126.17 (d, J=11.0 Hz).

IndCl-7-F (9) was obtained by reaction of 5-Ac-IndCl-7-F-4′-OMe with BF₃SMe₂ (12 eq.), and then K₂CO₃ following the same method as described to make IndBr-7-Br. ¹H NMR (499 MHz, CD₃OD) δ 7.41 (d, J=8.9 Hz, 1H), 6.95 (d, J=8.8 Hz, 2H), 6.72 (dd, J=12.1, 2.0 Hz, 1H), 6.61 (d, J=1.9 Hz, 1H). ¹⁹F NMR (470 MHz, CD₃OD) δ −129.01 (d, J=12.8 Hz). ESI (m/z, M+1) 279.0 (100%), 281.0 (31%).

### 10). Synthesis of 7-methyl-2-(4-hydroxyphenyl)-2H-indazol-5-ol (Ind-7-Me, 10)

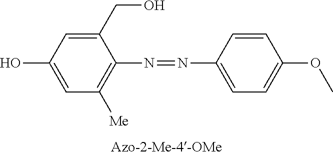 [SMILES: COc1ccc(N=Nc2c(C)cc(O)cc2CO)cc1]

3-Methyl-5-(hydroxymethyl)-4-((4-methoxyphenyl)diazenyl)phenol (AZO-2-Me-4′-OMe). Azo-2-Me-4′-OMe was obtained from the reaction of p-methoxyaniline (123.0 mg, 1.00 mmol) with NaNO₂ (72.0 mg, 1.03 mmol) and 3-methyl-5-hydroxymethyl phenol (138.0 mg, 1.00 mmol) in 78% yield as yellowish solid, following the same method as described to make Azo-2-Br-4′-OMe compound. ¹H NMR (499 MHz, CDCl₃) δ 7.85 (d, J=9.0 Hz, 2H), 7.02 (d, J=9.0 Hz, 2H), 6.84 (d, J=2.6 Hz, 1H), 6.81 (d, J=2.8 Hz, 1H), 4.36 (s, 2H), 3.90 (s, 3H), 2.62 (s, 3H). ¹³C NMR (126 MHz, CDCl₃) δ 162.14, 158.82, 146.99, 144.38, 142.69, 132.41, 124.56, 118.45, 116.77, 114.71, 63.89, 55.93, 19.47.

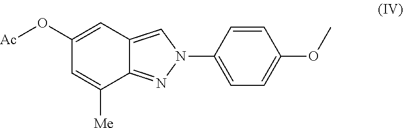 [SMILES: COc1ccc(-n2cc3cc(OC(C)=O)cc(C)c3n2)cc1]

5-Ac-Ind-7-Me-4′-OMe (IV) was obtained from the treatment of AZO-2-Me-4′-OMe (272.3 mg, 1.00 mmol) with pyridine HCl (800.0 mg) and acetic anhydride (1 mL) in 91% yield, following the same method as described to make 5-Ac-Ind-7-Br-4′-OMe. ¹H NMR (499 MHz, CDCl₃) δ 8.25 (s, 1H), 7.78 (d, J=9.1 Hz, 2H), 7.22 (d, J=1.7 Hz, 1H), 7.02 (d, J=9.2 Hz, 2H), 6.82 (d, J=1.7 Hz, 1H), 3.86 (s, 3H), 2.67 (s, 3H), 2.32 (s, 3H). ¹³C NMR (126 MHz, CDCl₃) δ 170.16, 159.44, 148.34, 145.96, 134.34, 130.00, 122.72, 121.85, 121.49, 121.18, 114.76, 108.23, 55.76, 21.31, 17.20.

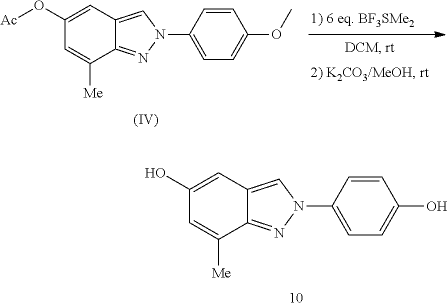 [SMILES: COc1ccc(-n2cc3cc(OC(C)=O)cc(C)c3n2)cc1 ; "(IV)" ; Cc1cc(O)cc2cn(-c3ccc(O)cc3)nc12]

7-Methyl-2-(4-hydroxyphenyl)-2H-indazol-5-ol (Ind-7-Me, 10). Ind-7-Me (10) was prepared from the treatment of 5-Ac-Ind-7-Me-4′-OMe (IV) (29.6 mg, 0.10 mmol) with BF₃—SMe₂ (20 eq.) and K₂CO₃ in 85% yield, following the same method as described to make Ind-7-Br (1). ¹H NMR (499 MHz, CDCl₃) δ 7.98 (s, 1H), 7.54 (d, J=8.9 Hz, 1H), 6.87 (d, J=8.9 Hz, 2H), 6.72 (d, J=1.2 Hz, 1H), 6.72 (s, 1H), 2.55 (s, 1H). ¹³C NMR (126 MHz, CDCl₃) δ 156.98, 151.95, 146.74, 133.55, 129.29, 123.14, 122.93, 120.27, 120.19, 116.23, 97.53, 17.20. ESI (m/z, M+1) 241.1 (100%).

### 11). Synthesis of 3-Chloro-7-methyl-2-(4-hydroxyphenyl)-2H-indazol-5-ol (IndCl-7-Me, 11)

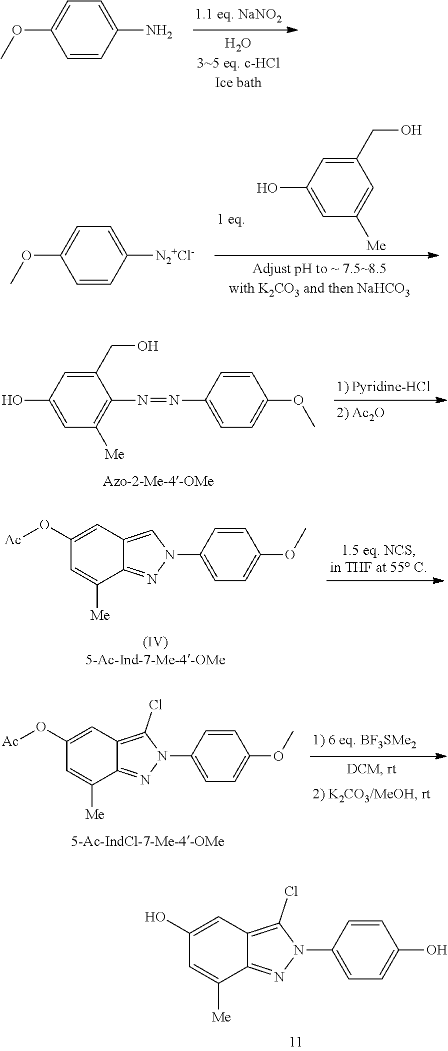

5-Ac-IndCl-7-Me-4′-OMe was obtained by reaction of 5-Ac-Ind-7-Me-4′-OMe (29.6 mg, 0.10 mmol) with NCS (20.0 mg, 0.15 mmol). ¹H NMR (499 MHz, CDCl₃) δ 7.59 (d, J=8.9 Hz, 2H), 7.19-7.18 (m, 1H), 7.07 (d, J=9.0 Hz, 2H), 6.87 (dd, J=2.2, 1.2 Hz, 1H), 3.90 (s, 3H), 2.63 (s, 3H), 2.35 (s, 3H). ¹³C NMR (126 MHz, CDCl₃) δ 170.21, 160.39, 147.11, 146.38, 131.74, 130.53, 127.43, 122.73, 120.51, 119.05, 114.56, 107.27, 55.86, 21.38, 16.82.

IndCl-7-Me (11) was obtained by reaction of 5-Ac-IndCl-7-Me-4′-OMe with BF₃—SMe₂ (20 eq.), and then K₂CO₃ following the same method as described to make IndBr-7-Br. ¹H NMR (499 MHz, CDCl₃) δ 7.33 (d, J=8.8 Hz, 2H), 6.87 (d, J=8.8 Hz, 2H), 6.73 (dd, J=2.3, 1.3 Hz, 1H), 6.57-6.57 (m, 1H), 2.46 (s, 3H). ¹³C NMR (126 MHz, CDCl₃) δ 158.02, 152.57, 145.21, 130.51, 129.56, 127.51, 121.39, 119.80, 118.78, 115.88, 95.91, 16.59. ESI (m/z, M+1) 275.0 (100%), 277.0 (29%).

### 12). Synthesis of 7-Bromo-3-chloro-2-(3-hydroxyphenyl)-2H-indazol-5-ol (IndCl-7-Br-3′-OH, 12)

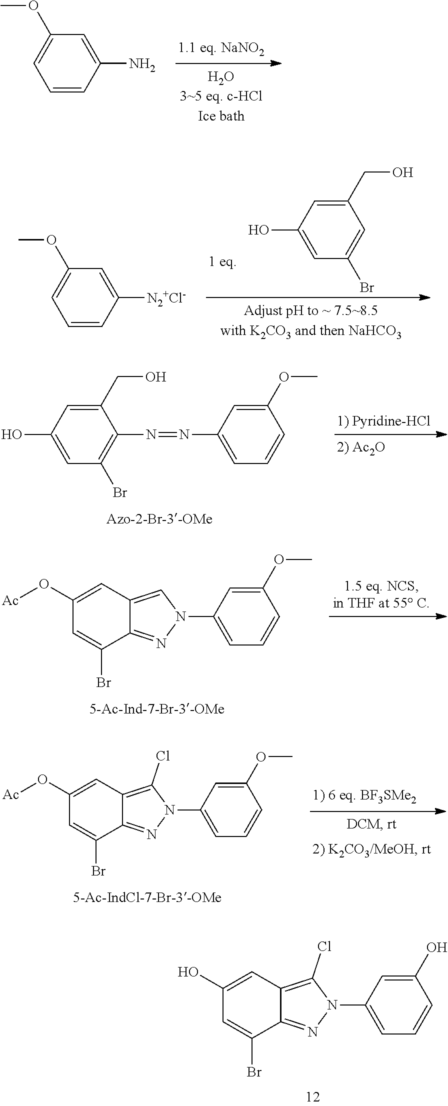

AZO-2-Br-3′-OMe was obtained from the reaction of 3-methoxyaniline (123.0 mg, 1.00 mmol) with NaNO₂ (72.0 mg, 1.03 mmol) and 3-bromo-5-hydroxymethyl phenol (203.0 mg, 1.00 mmol) in 91% yield as yellowish solid, following the same method as described to make Azo-2-Br-4′-OMe compound. ¹H NMR (499 MHz, CD₃OD) δ 7.52-7.49 (m, 1H), 7.42-7.38 (m, 2H), 7.21 (d, J=2.7 Hz, 1H), 7.02 (ddt, J=8.2, 2.6, 1.3 Hz, 1H), 6.92 (d, J=2.6 Hz, 1H), 4.49 (d, J=2.3 Hz, 2H), 3.86 (s, 3H).

5-Ac-Ind-7-Br-3′-OMe was obtained from the treatment of AZO-2-Br-3′-OMe (361.0 mg, 1.00 mmol) with pyridine HCl (950.0 mg) and acetic anhydride (1 mL) in 93% yield, following the same method as described to make 5-Ac-Ind-7-Br-4′-OMe.

5-Ac-IndCl-7-Br-3′-OMe was obtained by reaction of 5-Ac-Ind-7-Br-3′-OMe (36.1 mg. 0.10 mmol) with NCS (20.0 mg, 0.15 mmol). ¹H NMR (499 MHz, CDCl₃) δ 7.48 (t, J=8.1 Hz, 1H), 7.40 (d, J=2.0 Hz, 1H), 7.37 (d, J=1.9 Hz, 1H), 7.30-7.27 (m, 1H), 7.23 (t, J=2.3 Hz, 1H), 7.09 (ddd, J=8.4, 2.5, 0.9 Hz, 1H), 3.91 (s, 3H), 2.37 (s, 3H). ¹³C NMR (126 MHz, CDCl₃) δ 169.75, 160.36, 145.96, 145.55, 139.21, 130.17, 126.93, 121.72, 119.68, 118.42, 115.97, 112.74, 111.89, 109.75, 55.92, 21.30.

IndCl-7-Br-3′-OH (12) was obtained by reaction of 5-Ac-IndCl-7-Br-3′-OMe with BF₃—SMe₂ (20 eq.), and then K₂CO₃ following the same method as described to make IndBr-7-Br. ¹H NMR (499 MHz, CDCl₃-CD₃OD) δ 7.27 (t, J=8.1 Hz, 1H), 7.23 (d, J=2.1 Hz, 1H), 7.03 (dt, J=7.8, 1.0 Hz, 2H), 7.01 (t, J=2.1 Hz, 2H), 6.90 (ddd, J=8.2, 2.4, 1.0 Hz, 2H), 6.72 (d, J=2.1 Hz, 1H). ESI (m/z, M+1) 338.9 (100%), 340.9 (98%), 342.9 (29%).

### 13). Synthesis of 7-Chloro-2-(3-fluoro-4-hydroxyphenyl)-2H-indazol-5-ol (Ind-7-Cl-3′-F, 13)

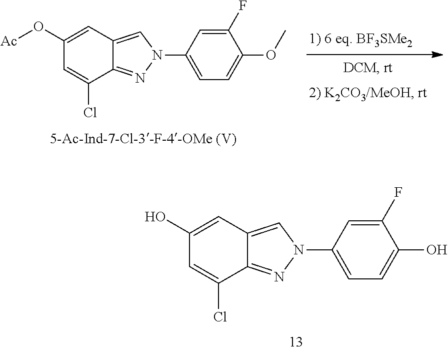 [SMILES: COc1ccc(-n2cc3cc(OC(C)=O)cc(Cl)c3n2)cc1F ; Oc1cc(Cl)c2nn(-c3ccc(O)c(F)c3)cc2c1]

Ind-7-Cl-3′-F (13) was prepared from the treatment of 5-Ac-Ind-7-Cl-3′-F-4′-OMe (V) (33.5 mg, 0.10 mmol) with BF₃—SMe₂ (25 eq.) and K₂CO₃ in 81% yield, following the same method as described to make Ind-7-Br (1). ¹H NMR (499 MHz, CD₃OD) δ 8.24 (s, 1H), 7.59 (dd, J=11.6, 2.6 Hz, 1H), 7.44 (dq, J=8.7, 2.6, 1.4 Hz, 1H), 7.04 (d, J=2.0 Hz, 1H), 6.85 (d, J=2.0 Hz, 1H), 7.03 (t, J=8.8 Hz, 1H). ¹⁹F NMR (470 MHz, CD₃OD) δ −135.58-135.97 (m). ESI (m/z, M+1) 279.03 (100%), 281.03 (31%).

### 14). Synthesis of 3,7-dichloro-2-(3-fluoro-4-hydroxyphenyl)-2H-indazol-5-ol (IndCl-7-Cl-3′-F, 14)

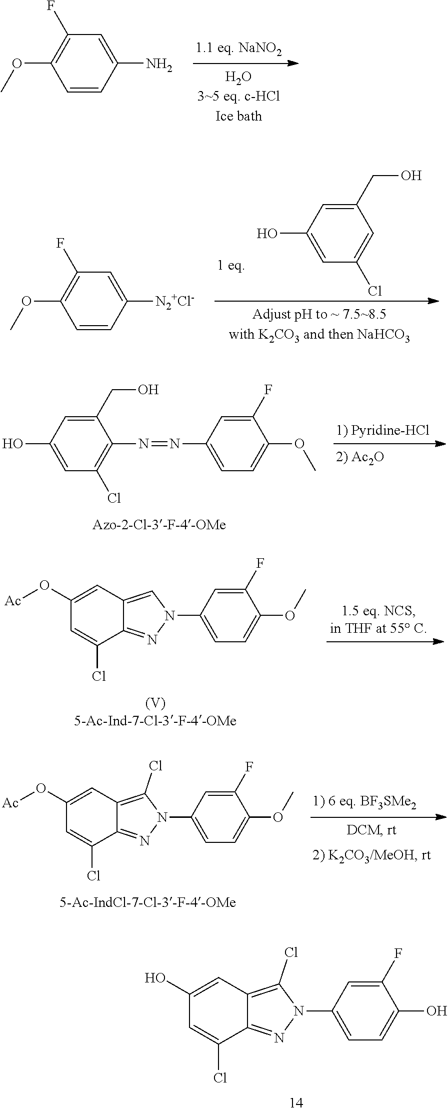

Azo-2-Cl-3′-F-4′-OMe was obtained from the reaction of 3-fluoro-4-methoxyaniline (141.0 mg, 1.00 mmol) with NaNO₂ (72.0 mg, 1.03 mmol) and 3-chloro-5-hydroxymethyl phenol (203.0 mg, 1.00 mmol) in 89% yield as yellowish solid, following the same method as described to make Azo-2-Br-4′-OMe compound. ¹H NMR (499 MHz, CD₃OD) δ 7.74-7.71 (m, 1H), 7.61 (dd, J=12.2, 2.3 Hz, 1H), 7.17 (t, J=8.6 Hz, 1H), 7.01 (d, J=2.7 Hz, 1H), 6.96 (d, J=2.7 Hz, 1H), 4.62 (s, 2H), 3.97 (s, 3H). ¹⁹F NMR (470 MHz, CD₃OD) δ −134.74 (dd, J=12.2, 8.6 Hz).

5-Ac-Ind-7-Cl-3′-F-4′-OMe was obtained from the treatment of AZO-2-Cl-3′-F-4′-OMe (311.0 mg, 1.00 mmol) with pyridine HCl (910.0 mg) and acetic anhydride (1 mL) in 88% yield, following the same method as described to make 5-Ac-Ind-7-Br-4′-OMe. ¹H NMR (499 MHz, CDCl₃) δ 8.38 (s, 1H), 7.74 (dd, J=11.6, 2.6 Hz, 1H), 7.67-7.64 (m, 1H), 7.38 (d, J=2.0 Hz, 1H), 7.17 (d, J=1.9 Hz, 1H), 7.11 (t, J=8.8 Hz, 1H), 3.99 (s, 3H), 2.36 (s, 3H). 19F NMR (470 MHz, CDCl₃) δ −132.14 (t, J=10.1 Hz).

IndCl-7-Cl-3′-F (14) was obtained in 73% yield via two steps from the reaction of 5-Ac-Ind-7-Cl-3′-F-4′-OMe (V) (33.5 mg, 0.10 mmol) with NCS (20.0 mg, 0.15 mmol), and subsequent treatment of the resultant 5-Ac-IndCl-7-Cl-3′-F-4′-OMe with BF₃—SMe₂ (25 eq.), and then K₂CO₃ following the same method as described to make IndBr-7-Br. ¹H NMR (499 MHz, CD₃OD) δ 7.38 (dd, J=11.1, 2.4 Hz, 1H), 7.28 (dq, J=8.9, 2.4, 1.4 Hz, 1H), 7.08 (t, J=8.9 Hz, 1H), 7.08 (d, J=2.1 Hz), 1H 7.01 (d, J=2.1 Hz, 1H). ¹⁹F NMR (470 MHz, CD₃OD) δ −136.57 (t, J=10.25 Hz). ESI (m/z, M+1) 312.9 (100%), 314.9 (51%).

### 15). Synthesis of 7-chloro-2-(2-fluoro-4-hydroxyphenyl)-2H-indazol-5-ol (Ind-7-Cl-2′-F, 15)

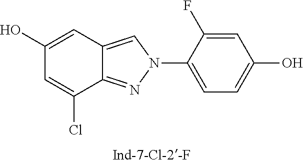 [SMILES: Oc1ccc(-n2cc3cc(O)cc(Cl)c3n2)c(F)c1]

Ind-7-Cl-2′-F (15) was prepared from the treatment of 5-Ac-Ind-7-Cl-2′-F-4′-OMe (VI) (33.5 mg, 0.10 mmol) with BF₃—SMe₂ (25 eq.) and K₂CO₃ in 85% yield, following the same method as described to make Ind-7-Br (1). ¹H NMR (499 MHz, CDCl₃) δ 8.13 (d, J=2.5 Hz, 1H), 7.67 (t, J=9.0 Hz, 1H), 7.06 (d, J=2.0 Hz, 1H), 6.85 (d, J=2.0 Hz, 1H), 6.74-6.66 (m, 2H). ¹⁹F NMR (470 MHz, CDCl₃) δ −123.76 (t, J=10.9 Hz). ESI (m/z, M+1) 297.0 (100%), 281.0 (29%).

### 16). Synthesis of 3,7-dichloro-2-(2-fluoro-4-hydroxyphenyl)-2H-indazol-5-ol (IndCl-7-Cl-2′-F, 16)

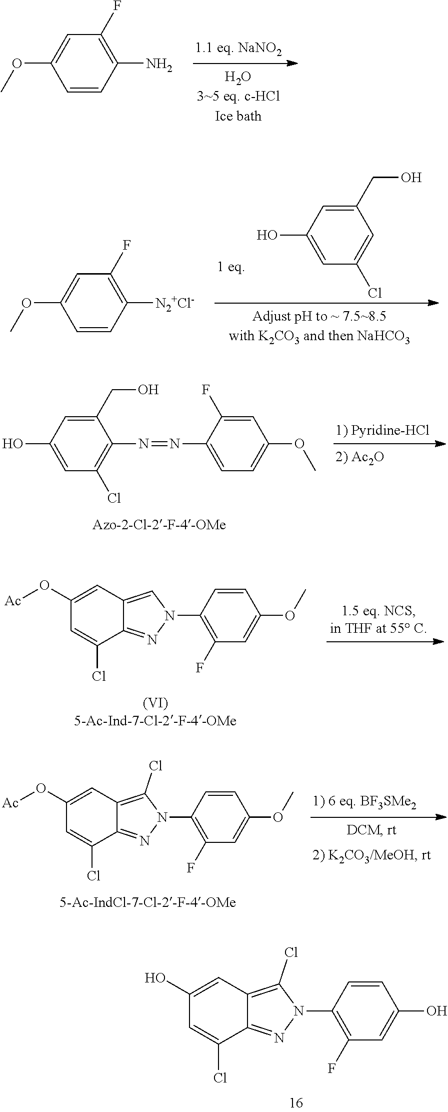

AZO-2-Cl-2′-F-4′-OMe was obtained from the reaction of 2-fluoro-4-methoxyaniline (141.0 mg, 1.00 mmol) with NaNO₂ (72.0 mg, 1.03 mmol) and 3-chloro-5-hydroxymethyl phenol (203.0 mg, 1.00 mmol) in 87% yield as yellowish solid, following the same method as described to make Azo-2-Br-4′-OMe compound. ¹H NMR (499 MHz, CD₃OD) δ 7.81 (t, J=8.1 Hz, 1H), 7.00 (d, J=2.6 Hz, 1H), 6.83 (d, J=2.6 Hz, 1H), 6.80-6.75 (m, 2H), 4.41 (s, 2H), 3.87 (s, 3H). ¹⁹F NMR (470 MHz, CD₃OD) δ −123.23 (dd, J=12.6, 8.7 Hz).

5-Ac-Ind-7-Cl-2′-F-4′-OMe was obtained from the treatment of AZO-2-Cl-2′-F-4′-OMe (310.0 mg, 1.00 mmol) with pyridine HCl (900.0 mg) and acetic anhydride (1 mL) in 91% yield, following the same method as described to make 5-Ac-Ind-7-Br-4′-OMe. ¹H NMR (500 MHz, CDCl₃) δ 8.43 (d, J=2.5 Hz, 1H), 7.95 (t, J=8.8 Hz, 1H), 7.37 (d, J=2.0 Hz, 1H), 7.17 (d, J=1.9 Hz, 1H), 6.86 (dd, J=9.2, 2.5 Hz, 1H), 6.82 (dd, J=12.8, 2.6 Hz, 1H), 3.88 (s, 3H), 2.34 (s, 3H). ¹⁹F NMR (470 MHz, CDCl₃) δ −122.58 (t, J=10.9 Hz). ¹³C NMR (126 MHz, CDCl₃) δ 169.86, 160.85, 158.34, 152.96, 148.52, 133.15, 126.43, 124.03, 123.11, 122.51, 121.73, 112.40, 110.33, 104.12, 58.40, 20.48.

IndCl-7-Cl-2′-F (16) was obtained in 71% yield via two steps from the reaction of 5-Ac-Ind-7-Cl-2′-F-4′-OMe (VI) (33.5 mg, 0.10 mmol) with NCS (20.0 mg, 0.15 mmol), and subsequent treatment of the resultant 5-Ac-IndCl-7-Cl-2′-F-4′-OMe with BF₃—SMe₂ (25 eq.), and then K₂CO₃ following the same method as described to make IndBr-7-Br. ¹H NMR (400 MHz, CDCl₃) δ 7.13 (t, J=8.8 Hz, 1H), 6.93 (d, J=2.0 Hz, 1H), 6.59-6.52 (m, 3H). ¹⁹F NMR (470 MHz, CD₃OD) δ −120.40 (t, J=9.8 Hz). ESI (m/z, M+1) 312.9 (100%), 314.9 (60%). 17). Synthesis of 3-chloro-6-fluoro-2-(4-hydroxyphenyl)-2H-indazol-5-ol (IndCl-6-F, 17)

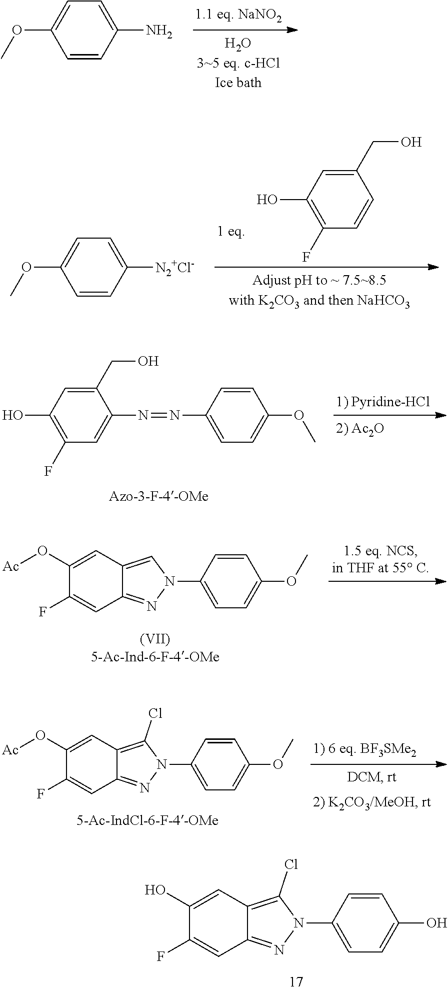

Azo-3-F-4′-OMe was obtained from the reaction of 4-methoxyaniline (123.0 mg, 1.00 mmol) with NaNO₂ (72.0 mg, 1.03 mmol) and 2-fluoro-5-hydroxymethyl phenol (138.0 mg, 0.97 mmol) in 89% yield as yellowish solid, following the same method as described to make Azo-2-Br-4′-OMe compound. ¹H NMR (499 MHz, CDCl₃) δ 7.83 (d, J=9.0 Hz, 2H), 7.63 (d, J=11.7 Hz, 1H), 7.12 (d, J=8.8 Hz, 1H), 7.01 (d, J=8.9 Hz, 2H), 5.00 (s, 2H), 3.90 (s, 2H). 19F NMR (470 MHz, CDCl₃) δ −140.33.

5-Ac-Ind-6-F-4′-OMe (VII) was obtained from the treatment of Azo-3-F-4′-OMe (276.3 mg, 1.00 mmol) with pyridine HCl (800.0 mg) and acetic anhydride (1 mL) in 91% yield, following the same method as described to make 5-Ac-Ind-7-Br-4′-OMe. ¹H NMR (499 MHz, CDCl₃) δ 8.29 (s, 1H), 7.75 (d, J=9.1 Hz, 2H), 7.47 (d, J=10.6 Hz, 1H), 7.43 (d, J=7.4 Hz, 1H), 7.03 (d, J=9.0 Hz, 2H), 3.88 (s, 3H), 2.37 (s, 3H). ¹⁹F NMR (470 MHz, CDCl₃) δ −127.64 (dd, J=10.7, 7.6 Hz).

IndCl-6-F (17) was obtained in 76% yield via two steps from the reaction of 5-Ac-Ind-6-F-4′-OMe (VII) (30.0 mg, 0.10 mmol) with NCS (20.0 mg, 0.15 mmol), and subsequent treatment of the resultant 5-Ac-IndCl-6-F-4′-OMe with BF₃—SMe₂ (25 eq.), and then K₂CO₃ following the same method as described to make IndBr-7-Br. ¹H NMR (499 MHz, CD₃OD) δ 7.39 (d, J=8.4 Hz, 21H), 7.23 (d, J=11.0 Hz, 1H), 6.95 (d, J=8.2 Hz, 2H), 6.92 (d, J=8.4 Hz, 1H). ¹⁹F NMR (470 MHz, CDCl₃) δ −130.34 (t, J=8.6 Hz). ESI (m/z, M+1) 279.0 (100%), 281.0 (28%).

### 18). Synthesis of 3-Chloro-6-fluoro-2-(4-hydroxy-2-methylphenyl)-2H-indazol-5-ol (IndCl-6-F-2′-Me, 18)

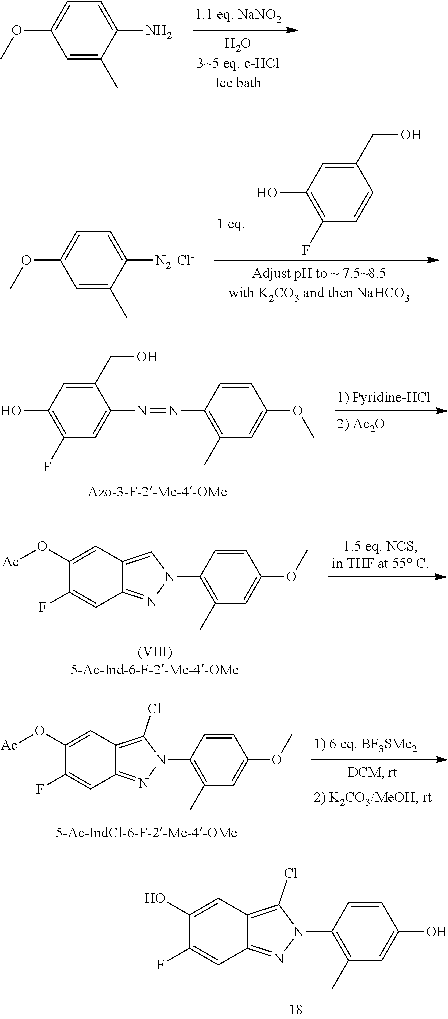

Azo-3-F-2′-Me-4′-OMe was obtained from the reaction of 2-methyl-4-methoxyaniline (137.0 mg, 1.00 mmol) with NaNO₂ (72.0 mg, 1.03 mmol) and 2-fluoro-5-hydroxymethyl phenol (138.0 mg, 0.97 mmol) in 78% yield as yellowish solid, following the same method as described to make Azo-2-Br-4′-OMe compound. ¹H NMR (499 MHz, CDCl₃) δ 7.80 (d, J=9.0 Hz, 1H), 7.59 (d, J=11.60 Hz, 1H), 7.58 (d, J=8.5 Hz, 1H), 7.15 (d, J=8.8 Hz, 1H), 6.84 (d, J=8.2 Hz, 1H), 5.06 (s, 2H), 3.90 (s, 3H), 2.65 (d, J=0.9 Hz, 3H). ¹⁹F NMR (470 MHz, CDCl₃) δ −140.68 (t, J=10.4 Hz).

5-Ac-Ind-6-F-2′-Me-4′-OMe (VIII) was obtained from the treatment of Azo-3-F-2′-Me-4′-OMe (290.3 mg, 1.00 mmol) with pyridine HCl (900.0 mg) and acetic anhydride (1 mL) in 88% yield, following the same method as described to make 5-Ac-Ind-7-Br-4′-OMe. ¹H NMR (499 MHz, CDCl₃) δ 8.03 (s, 1H), 7.47 (d, J=11.4 Hz, 1H), 7.45 (d, J=8.2 Hz, 1H), 7.29 (d, J=8.5 Hz, 1H), 6.86 (d, J=2.7 Hz, 1H), 6.83 (dd, J=8.6, 2.9 Hz, 1H), 3.85 (s, 3H), 2.37 (s, 3H), 2.17 (s, 3H). ¹⁹F NMR (470 MHz, CDCl₃) δ −128.17 (dd, J=11.1, 7.4 Hz).

IndCl-6-F-2′-Me (18) was obtained in 76% yield via two steps from the reaction of 5-Ac-Ind-6-F-2′-Me-4′-OMe (VIII) (30.0 mg, 0.10 mmol) with NCS (20.0 mg, 0.15 mmol), and subsequent treatment of the resultant 5-Ac-IndCl-6-F-2′-Me-4′-OMe with BF₃—SMe₂ (25 eq.), and then K₂CO₃ following the same method as described to make IndBr-7-Br. ¹H NMR (499 MHz, CD₃OD) δ 7.27 (d, J=11.1 Hz, 1H), 7.15 (d, J=8.5 Hz, 1H), 6.95 (d, J=8.4 Hz, 1H), 6.83 (d, J=2.7 Hz, 1H), 6.78 (dd, J=8.5, 2.6 Hz, 1H). ¹⁹F NMR (470 MHz, CDCl₃) δ −130.34 (dd, J=11.1, 7.4 Hz). ESI (m/z, M+1) 293.0 (100%), 295.0 (27%).

### 19). Synthesis of 3,6-dichloro-2-(4-hydroxyphenyl)-2/I-indazol-5-ol (IndCl-6-Cl, 19)

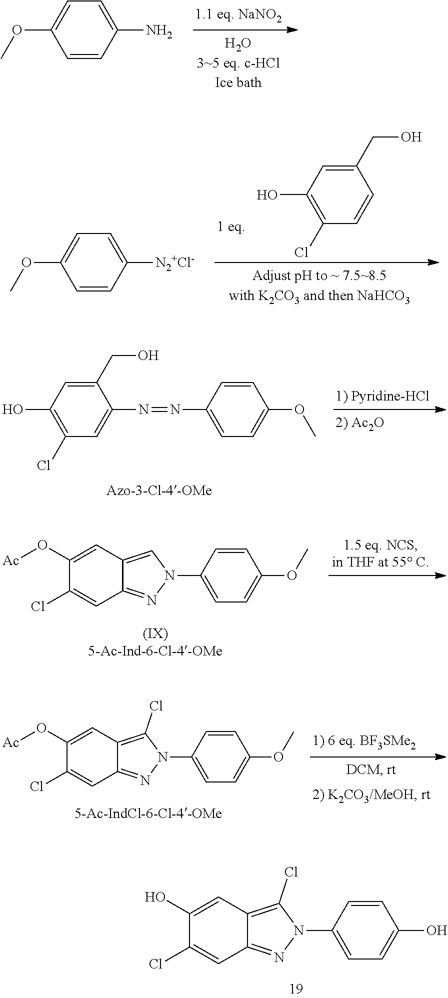

Azo-3-Cl-4′-OMe was obtained from the reaction of 4-methoxyaniline (123.0 mg, 1.00 mmol) with NaNO₂ (72.0 mg, 1.03 mmol) and 2-chloro-5-hydroxymethyl phenol (158.6 mg, 0.97 mmol) in 82% yield as yellowish solid, following the same method as described to make Azo-2-Br-4′-OMe compound. ¹H NMR (499 MHz, CDCl₃) δ 7.89 (s, 1H), 7.85 (d, J=9.0 Hz, 2H), 7.15 (s, 1H), 7.02 (d, J=9.0 Hz, 2H), 5.01 (s, 3H), 3.91 (s, 3H). ¹³C NMR (126 MHz, CDCl₃) δ 162.63, 153.51, 146.92, 144.67, 139.97, 125.03, 120.65, 118.89, 116.14, 114.70, 63.04, 55.91.

5-Ac-Ind-6-Cl-4′-OMe (IX) was obtained from the treatment of Azo-3-Cl-4′-OMe (293.0 mg, 1.00 mmol) with pyridine HCl (900.0 mg) and acetic anhydride (1 mL) in 90% yield, following the same method as described to make 5-Ac-Ind-7-Br-4′-OMe. ¹H NMR (499 MHz, CDCl₃) δ 8.10 (s, 1H), 7.69 (d, J=0.8 Hz, 1H), 7.57 (d, J=9.0 Hz, 2H), 7.07 (d, J=0.9 Hz, 1H), 6.84 (d, J=9.0 Hz, 2H), 3.69 (s, 3H), 2.20 (s, 3H). ¹³C NMR (126 MHz, CDCl₃) δ 169.60, 159.88, 147.82, 142.32, 133.95, 127.46, 122.73, 121.24, 121.12, 119.07, 115.01, 113.43, 55.91, 20.92.

IndCl-6-Cl (19) was obtained in 76% yield via two steps from the reaction of 5-Ac-Ind-6-Cl-4′-OMe (IX) (31.7 mg, 0.10 mmol) with NCS (20.0 mg, 0.15 mmol), and subsequent treatment of the resultant 5-Ac-IndCl-6-Cl-4′-OMe with BF₃—SMe₂ (25 eq.), and then K₂CO₃ following the same method as described to make IndBr-7-Br. ¹H NMR (499 MHz, CD₃OD) δ 7.64 (s, 1H), 7.42 (d, J=8.7 Hz, 2H), 6.95 (d, J=8.7 Hz, 2H), 6.91 (s, 1H). ¹³C NMR (126 MHz, CDCl₃) δ 162.81, 153.20, 147.92, 133.96, 131.67, 131.09, 122.67, 122.34, 121.81, 119.49, 103.14. ESI (m/z, M+1) 295.0 (100%), 297.0 (53%).

### 20). Synthesis of 3,6-dichloro-2-(4-hydroxy-2-methylphenyl)-2H-indazol-5-ol (IndCl-6-Cl-2′-Me, 20)

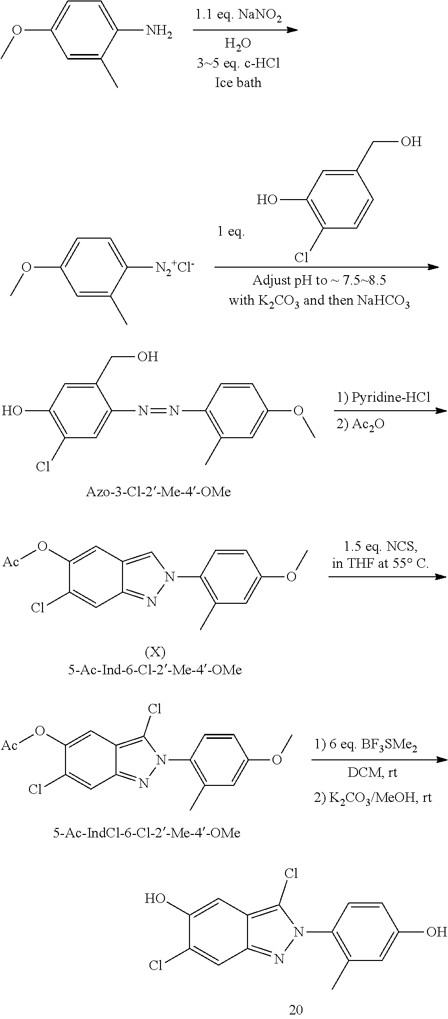

Azo-3-Cl-2′-Me-4′-OMe was obtained from the reaction of 2-methyl-4-methoxyaniline (137.0 mg, 1.00 mmol) with NaNO₂ (72.0 mg, 1.03 mmol) and 2-chloro-5-hydroxymethyl phenol (159.0 mg, 0.97 mmol) in 68% yield as yellowish solid, following the same method as described to make Azo-2-Br-4′-OMe compound. ¹H NMR (499 MHz, CDCl₃) δ 7.82 (s, 1H), 7.58 (d, J=8.9 Hz, 1H), 7.14 (s, 1H), 6.84 (d, J=3.0 Hz, 1H), 6.79 (dd, J=9.0, 2.8 Hz, 1H), 5.03 (s, 3H), 3.87 (s, 4H), 2.73 (s, 4H).

5-Ac-Ind-6-Cl-2′-Me-4′-OMe (X) was obtained from the treatment of Azo-3-Cl-2′-Me-4′-OMe (307.0 mg, 1.00 mmol) with pyridine HCl (950.0 mg) and acetic anhydride (1 mL) in 81% yield, following the same method as described to make 5-Ac-Ind-7-Br-4′-OMe. ¹H NMR (499 MHz, CDCl₃) δ 8.03 (d, J=1.0 Hz, 1H), 7.89 (d, J=0.8 Hz, 1H), 7.47 (s, 1H), 7.30 (d, J=8.6 Hz, 1H), 6.87 (d, J=2.9 Hz, 1H), 6.83 (dd, J=8.6, 2.8 Hz, 1H), 3.86 (s, 3H), 2.39 (s, 3H), 2.17 (s, 3H). ¹³C NMR (126 MHz, CDCl₃) δ 169.72, 160.37, 147.47, 142.18, 135.72, 133.44, 127.84, 127.27, 125.55, 120.30, 119.16, 116.50, 113.45, 111.98, 55.82, 20.93, 18.23.

IndCl-6-Cl-2′-Me (20) was obtained in 73% yield via two steps from the reaction of 5-Ac-Ind-6-Cl-2′-Me-4′-OMe (X) (33.0 mg, 0.10 mmol) with NCS (20.0 mg, 0.15 mmol), and subsequent treatment of the resultant 5-Ac-IndCl-6-Cl-2′-Me-4′-OMe with BF₃—SMe₂ (25 eq.), and then K₂CO₃ following the same method as described to make IndBr-7-Br. ¹H NMR (499 MHz, CD₃OD) δ 7.66 (s, 1H), 7.14 (d, J=8.5 Hz, 1H), 6.95 (s, 1H), 6.82 (d, J=2.8 Hz, 1H), 6.77 (dd, J=8.5, 2.7 Hz, 1H). ¹³C NMR (126 MHz, CD₃OD) δ 159.92, 149.86, 144.68, 137.88, 129.52, 129.30, 128.43, 120.74, 118.52, 117.59, 113.90, 99.87, 30.03, 16.77. ESI (m/z, M+1) 309.0 (100%), 311.0 (56%).

### 21) Synthesis of 3-chloro-2-(4-hydroxyphenyl)-2H-indazole-5,7-diol (IndCl-7-OH, 21)

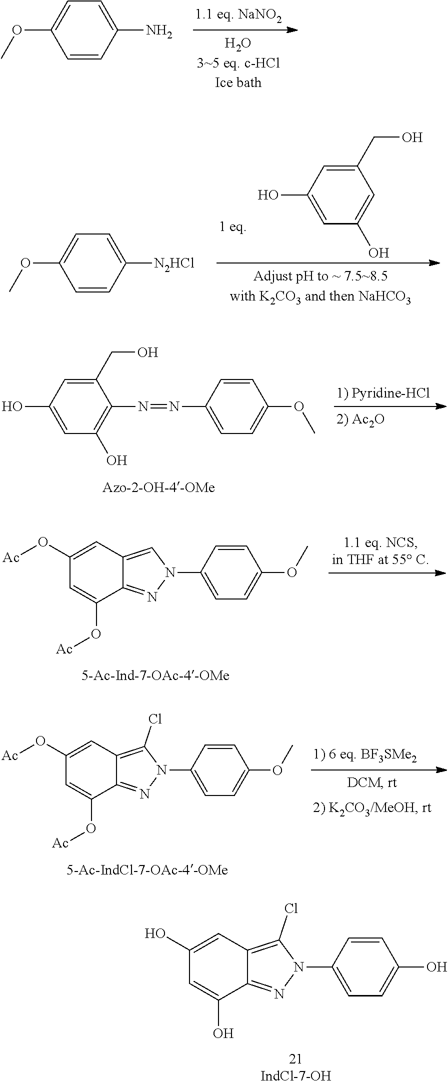

Azo-2-OH-4′-OMe was obtained from the reaction of 4-methoxyaniline (123.0 mg, 1.00 mmol) with NaNO₂ (72.0 mg, 1.03 mmol) and 3-hydroxy-5-hydroxymethyl phenol (140.1 mg, 0.97 mmol) in 86% yield as yellowish solid, following the same method as described to make Azo-2-Br-4′-OMe compound. ¹H NMR (500 MHz, CDCl₃) δ 7.66 (d, J=8.9 Hz, 2H), 6.96-6.93 (m, 2H), 6.52 (d, J=2.6 Hz, 1H), 6.24 (d, J=2.6 Hz, 1H), 4.99 (s, 2H), 3.84 (s, 3H). ¹³C NMR (126 MHz, CD₃OD) δ 162.79, 161.45, 157.06, 145.24, 144.50, 129.08, 122.96, 114.60, 107.54, 102.11, 60.60, 55.33.

5-Ac-Ind-7-OAc-4′-OMe was obtained from the treatment of Azo-2-OH-4′-OMe (274.3 mg, 1.00 mmol) with pyridine HCl (800.0 mg) and acetic anhydride (1 mL) in 88% yield, following the same method as described to make 5-Ac-Ind-7-Br-4′-OMe. ¹H NMR (500 MHz, CDCl₃) δ 8.32 (s, 1H), 7.75 (d, J=9.00, 2H), 7.40 (s, 1H), 7.02 (d, J=9.00, 2H), 3.88 (s, 3H), 2.52 (s, 3H), 2.39 (s, 3H). ¹³C NMR (126 MHz, CDCl₃) δ 169.33, 167.98, 159.97, 142.44, 142.21, 138.28, 133.77, 123.01, 122.21, 121.32, 119.71, 114.89, 111.19, 55.89, 20.90.

IndCl-7-OH (21) was obtained in 65% yield via two steps from the reaction of 5-Ac-Ind-7-OAc-4′-OMe (34.0 mg, 0.10 mmol) with NCS (14.6 mg, 0.11 mmol), and subsequent treatment of the resultant 5-Ac-IndCl-7-OAc-4′-OMe with BF₃—SMe₂ (25 eq.), and then K₂CO₃ following the same method as described to make IndBr-7-Br.

5-Ac-IndCl-7-OAc-4′-OMe: ¹H NMR (500 MHz, CDCl₃) δ 7.57 (d, J=8.9 Hz, 2H), 7.28 (d, J=1.9 Hz, 1H), 7.05 (d, J=8.9 Hz, 2H), 6.98 (d, J=2.0 Hz, 1H), 3.90 (s, 3H), 2.42 (s, 3H), 2.34 (s, 3H). ¹³C NMR (126 MHz, CDCl₃) δ 169.71, 168.85, 160.56, 145.53, 141.15, 140.73, 131.37, 127.46, 121.45, 120.67, 115.63, 114.53, 108.01, 55.89, 21.34.

IndCl-7-OH (21): ¹H NMR (500 MHz, CD₃OD) δ 7.59 (s, 1H), 7.38 (d, J=8.8 Hz, 2H), 6.93 (s,/H), 6.88 (d, J=8.8 Hz, 2H). ESI (m/z, M+1) 277.0. HRMS (ESI, M+1) Calcd. Mass for C₁₃H₁₀ClN₂O₃ 277.0380, found 277.0370.

### Synthesis of 3-chloro-2-(4-hydroxy-2-methylphenyl)-2H-indazole-5,7-diol (22)

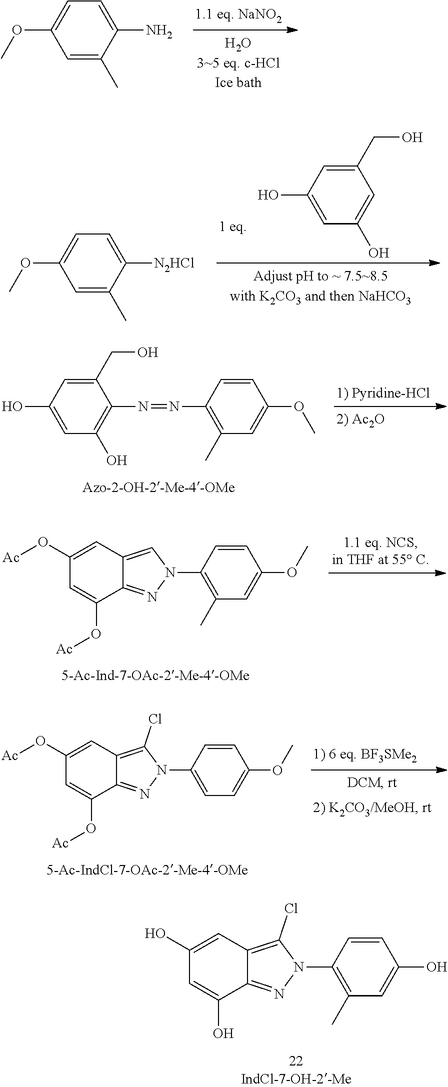

Azo-2-OH-2′-Me-4′-OMe was obtained from the reaction of 2-methyl-4-methoxyaniline (137.0 mg, 1.00 mmol) with NaNO₂ (72.0 mg, 1.03 mmol) and 3-hydroxy-5-hydroxymethyl phenol (140.1 mg, 0.97 mmol) in 87% yield as yellowish solid, following the same method as described to make Azo-2-Br-4′-OMe compound. ¹H NMR (499 MHz, CD₃OD) δ 7.68 (d, J=8.6 Hz, 1H), 6.81 (d, J=2.7 Hz, 1H), 6.79 (s, 1H), 6.56 (s, 1H), 6.24 (d, J=2.6 Hz, 1H), 5.04-5.01 (m, 2H), 3.82 (s, 1H), 2.50 (s, 3H). ¹³C NMR (126 MHz, CD₃OD) δ 162.94, 161.08, 158.14, 142.08, 136.54, 130.03, 117.26, 115.79, 113.23, 108.68, 108.10, 102.54, 61.67, 55.61, 18.26.

5-Ac-Ind-7-OAc-2′-Me-4′-OMe was obtained from the treatment of Azo-2-OH-2′-Me-4′-OMe (288.3 mg, 1.00 mmol) with pyridine HCl (800.0 mg) and acetic anhydride (1 mL) in 78% yield, following the same method as described to make 5-Ac-Ind-7-Br-4′-OMe. ¹H NMR (499 MHz, CDCl₃) δ 8.09 (s, 1H), 7.37 (d, J=1.9 Hz, 1H), 7.34 (d, J=8.6 Hz, 1H), 6.98 (d, J=1.9 Hz, 1H), 6.88 (d, J=2.8 Hz, 1H), 6.85 (dd, J=8.6, 2.8 Hz, 1H), 3.87 (s, 3H), 2.43 (s, 3H), 2.35 (s, 3H), 2.20 (s, 3H). ¹³C NMR (126 MHz, CDCl₃) δ 169.95, 169.01, 160.37, 145.10, 141.57, 141.18, 135.98, 133.53, 128.02, 126.12, 122.88, 116.46, 114.34, 111.90, 109.13, 55.81, 21.41, 21.39, 18.25.

IndCl-7-OH-2′-Me (22) was obtained in 63% yield via two steps from the reaction of 5-Ac-Ind-7-OAc-2′-Me-4′-OMe (35.4 mg, 0.10 mmol) with NCS (14.6 mg, 0.11 mmol), and subsequent treatment of the resultant 5-Ac-IndCl-7-OAc-2′-Me-4′-OMe with BF₃—SMe₂ (25 eq.), and then K₂CO₃ following the same method as described to make IndBr-7-Br.

5-Ac-IndCl-7-OAc-2′-Me-4′-OMe: ¹H NMR (499 MHz, CDCl₃) δ 7.29 (d, J=2.6 Hz, 1H), 7.27 (d, J=8.4 Hz, 1H), 7.02 (d, J=1.9 Hz, 1H), 6.89 (d, J=2.8 Hz, 1H), 6.86 (dd, J=8.5, 2.9 Hz, 1H), 3.87 (s, 3H), 2.40 (s, 3H), 2.35 (s, 3H), 2.05 (s, 3H).

IndCl-7-OH-2′-Me (22): ¹H NMR (499 MHz, CD₃OD) δ 7.74 (s, 1H), 7.15 (d, J=8.3 Hz, 3H), 6.81 (s, 3H), 6.77 (d, J=8.3 Hz, 4H), 6.43 (s, 1H), 2.02 (d, J=2.0 Hz, 3H). ESI (m/z, M+1) 291.0.

### 6. Biology Examples

### Example 1. Relative Binding Affinity (RBA) Assay in ERα and ERβ

The methodology for this radiometric competitive binding assay, using tritiated estradiol as tracer and purified, full length human ERα and ERβ, has been described (De Angelis et al., 2005). All values are a relative binding affinity, where E2 (estradiol) is 100%. This corresponds to a K_{d} of 0.2 nM for ERα and 0.5 nM for ERβ from which the Kᵢ values for the individual compounds can be calculated.

TABLE 1
Relative ERα and ERβ binding affinities and ERβ/ERα selectivity for 7-substitute
indazole analogs.

| entry | | | RBA | | |
|---|---|---|---|---|---|
| # | Code | Structure | ERα | ERβ | β/α |
| 1 | Ind-7-Br | 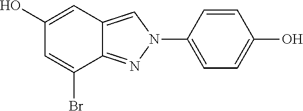 [SMILES: Oc1ccc(-n2cc3cc(O)cc(Br)c3n2)cc1] | 0.244 | 32.01 ± 0.09 | 131 |
| 2 | IndF-7-Br | 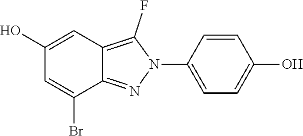 [SMILES: Oc1ccc(-n2nc3c(Br)cc(O)cc3c2F)cc1] | 2.27 | 159.03 | 70.1 |
| 3 | IndCI-7-Br | 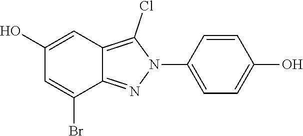 [SMILES: Oc1ccc(-n2nc3c(Br)cc(O)cc3c2Cl)cc1] | 15.57 | 282 ± 23 | 18 |
| 4 | IndBr-7-Br | 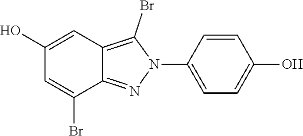 [SMILES: Oc1ccc(-n2nc3c(Br)cc(O)cc3c2Br)cc1] | 21.29 | 153 ± 33 | 7 |
| 5 | IndI-7-Br | 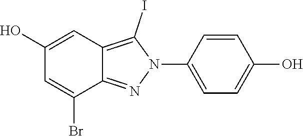 [SMILES: Oc1ccc(-n2nc3c(Br)cc(O)cc3c2I)cc1] | 6.95 | 159 | 23 |
| 6 | Ind-7-Cl | 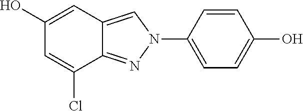 [SMILES: Oc1ccc(-n2cc3cc(O)cc(Cl)c3n2)cc1] | 0.098 | 19.6 ± 6.1 | 200 |
| 7 | IndCI-7-CI | 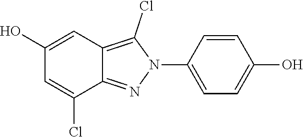 [SMILES: Oc1ccc(-n2nc3c(Cl)cc(O)cc3c2Cl)cc1] | 6.9 ± 2.4 | 313 ± 35 | 45 |
| 8 | Ind-7-F | 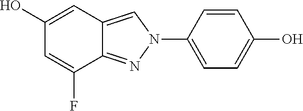 [SMILES: Oc1ccc(-n2cc3cc(O)cc(F)c3n2)cc1] | 0.258 | 22.4 | 87 |
| 9 | IndCI-7-F | 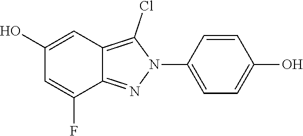 [SMILES: Oc1ccc(-n2nc3c(F)cc(O)cc3c2Cl)cc1] | 2.2 | 82.3 | 37 |
| 10 | Ind-7-Me | 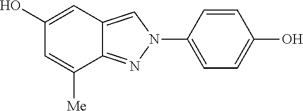 [SMILES: Cc1cc(O)cc2cn(-c3ccc(O)cc3)nc12] | 0.018 | 2.59 | 144 |
| 11 | IndCI-7-Me | 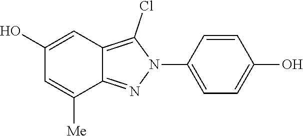 [SMILES: Cc1cc(O)cc2c(Cl)n(-c3ccc(O)cc3)nc12] | 2.76 | 45.9 | 17 |
| 12 | IndCI-7-Br-3′- OH | 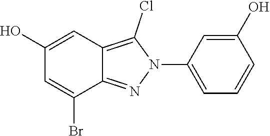 [SMILES: Oc1cccc(-n2nc3c(Br)cc(O)cc3c2Cl)c1] | 0.16 | 39.4 | 246 |
| 13 | Ind-7-Cl-3′-F | 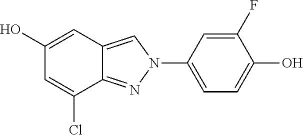 [SMILES: Oc1cc(Cl)c2nn(-c3ccc(O)c(F)c3)cc2c1] | 0.023 | 10.76 | 469 |
| 14 | IndCI-7-Cl-3′-F | 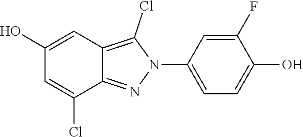 [SMILES: Oc1cc(Cl)c2nn(-c3ccc(O)c(F)c3)c(Cl)c2c1] | 0.86 | 32.96 | 38.3 |
| 15 | Ind-7-C1-2′-F | 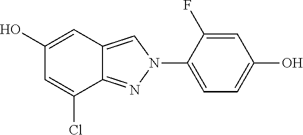 [SMILES: Oc1ccc(-n2cc3cc(O)cc(Cl)c3n2)c(F)c1] | 0.052 | 9.63 | 185 |
| 16 | IndCl-7-Cl-2′-F | 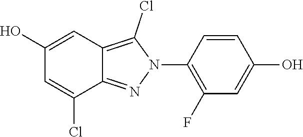 [SMILES: Oc1ccc(-n2nc3c(Cl)cc(O)cc3c2Cl)c(F)c1] | 4.71 | 31.76 | 6.74 |
| 21 | IndCI-7-OH | 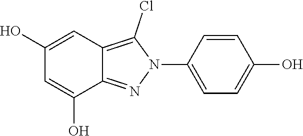 [SMILES: Oc1ccc(-n2nc3c(O)cc(O)cc3c2Cl)cc1] | 0.001 | 0.051 | 51 |
| 22 | IndCl-7-OH-2′- Me | 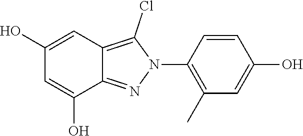 [SMILES: Cc1cc(O)ccc1-n1nc2c(O)cc(O)cc2c1Cl] | 0.0005 | 0.006 | 12 |

Many of the indazoles that have a substituent at the 7-position of the indazole show very high binding selectivity for ERβ vs. ERα (Table 1). The RBA for ERβ of compounds 3 (IndCl-7-Br) and 7 (IndCl-7-Cl) was measured to be about 300 compared to the value of 100 for estradiol. RBA toward ERβ for compounds 2 (IndF-7-Br), 4 (IndBr-7-Br), and 5 (IndI-7-Br) were measured at around 150. The selectivity of binding to ERβ vs ERα for compounds 6, 12, and 13 was 200-, 246-, and 469-fold, respectively. The ERβ selectivity of compounds 1, 10, and 15 was 131-, 144-, and 185-fold, respectively. All other compounds also exert good ERB selectivity. Compounds with the hydroxy group at 7-position (compound 21, IndCl-7-OH, and compound 22, IndCl-7-OH-2′-Me) exhibited lower binding affinity for both ERβ and ERα, but the specificity in binding to ERβ over ERα for compound 21 and 22 remains consistent at 51 and 12, respectively.

TABLE 2
Relative ERα and ERβ binding affinities and ERβ/ERα selectivity for 6-substituted
indazole analogs.

| | | | RBA | | |
|---|---|---|---|---|---|
| entry # | Code | Structure | ERα | ERβ | β/α |
| 17 | IndCI-6-F | 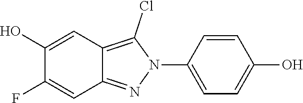 [SMILES: Oc1ccc(-n2nc3cc(F)c(O)cc3c2Cl)cc1] | 0.968 | 47.11 | 48.7 |
| 18 | IndCI-6-F-2′-Me | 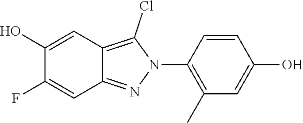 [SMILES: Cc1cc(O)ccc1-n1nc2cc(F)c(O)cc2c1Cl] | 0.545 | 2.53 | 4.64 |
| 19 | IndCI-6-CI | 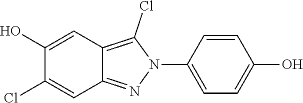 [SMILES: Oc1ccc(-n2nc3cc(Cl)c(O)cc3c2Cl)cc1] | 0.055 | 0.852 | 15.49 |
| 20 | IndCI-6-Cl-2′-Me | 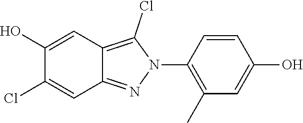 [SMILES: Cc1cc(O)ccc1-n1nc2cc(Cl)c(O)cc2c1Cl] | 0.071 | 0.187 | 2.63 |

The indazoles that have a substituent at 6-position show a good to moderate ERB selectivity (Table 2).

### Example 2. In Vitro Cell Assay

Primary OPC Cultures: Primary OPCs, isolated from postnatal day P1 C57BL/6 male and female mouse cortices as described previously, are treated with 10 nM ligands in differentiating medium for 3 days (Tiwari-Woodruff et al., 2001; Tiwari-Woodruff et al., 2006). Primary OPC are cultured into 8-well chamber slides (three wells per condition, 2.5×10⁵ cells/well) for three days to attach and five days in differentiating media (consisting of DMEM-F12 with triiodothyronine- and thyroxine-containing Sato as well as penicillin, streptomycin, insulin, N-acetyl-L-cysteine, forskolin, ciliary neurotrophic factor, neurotrophin-3, and platelet-derived growth factor receptor α (Tiwari-Woodruff et al., 2001)). A positive control (IndC1), a negative control (Vehicle consisting of the media+EtOH mixture used to dissolve IndCl), and a normal control (differentiating media alone) are used for comparison. At the end of the treatment period, cells are fixed, stained by immunocytochemistry (primary antibody polyclonal chicken myelin basic protein (MBP, Millipore AB9348), and co-stained with nuclear stain-DAPI), and imaged with an Olympus BX61 confocal microscope (Olympus America Inc., Center Valley, PA) at 10× magnification (3 images per well). Cells are counted using the ImageJ multipoint tool, and counts are then divided by the image area (mm²). Average cell density for each condition is then divided by the normal condition cell density. Analysis of OL differentiation consists of counting the number of MBP+ cells and process extensions that are longer than the respective cell-body diameter and tracking the number of highly branched MBP+ cells (with three or more processes) (Monnerie et al., 2017). Statistics are performed using GraphPad Prism 6 Software (La Jolla, CA). One-way ANOVA with Tukey's posthoc test for multiple comparisons is used to generate p-values, and data are presented as mean±SEM (with α≤0.05).

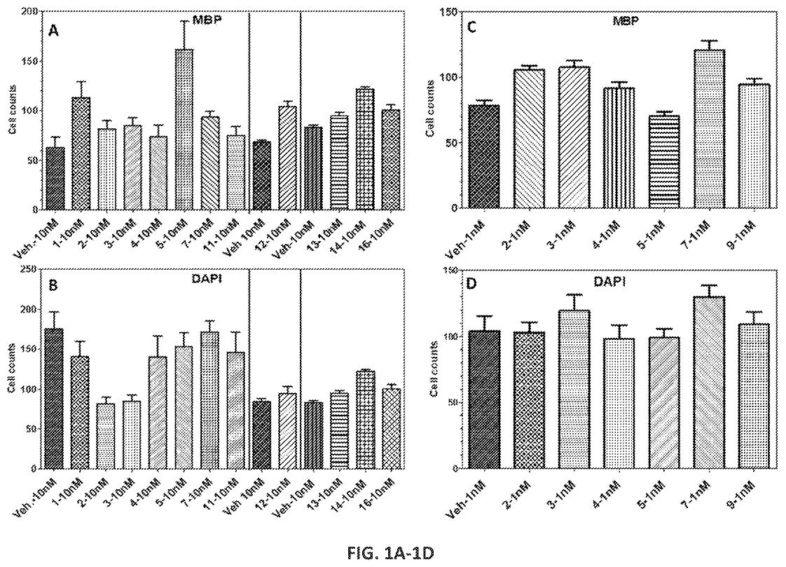

FIG. 1A-1D show the effects of treatment with the 7-substituted indazole compounds on the expression of myelin basic protein in OLs (MBP⁺-OLs, top two panels) as an indication of murine oligodendrocyte precursor cell (OPC) differentiation, as well as the count of the total cells as an indication of cell proliferation/survival (DAPI nucleus stain, bottom two panels). The cells were treated with the listed compounds at 10 nM (FIG. 1A, 1B) and at 1 nM (FIG. 1C, 1D). Compound 7 (IndCl-7-C₁) exerts a particularly significant increase in MBP expression level at 1 nM.

FIGS. 1A and 1C show bar graphs quantifying the number of MBP+ (oligodendrocytes) murine OLs for each treatment group in Table 1. Analogues Ind-7-Br (compound 1), IndI-7-Br (compound 5), IndCl-7-Cl (compound 7), and IndCl-7-Cl-3′-F (compound 14) showed a significant increase in the number of MBP+OLs that had an increase in the percentage of branched OLs, compared to vehicle-treated cells at 10 nM. Especially, compounds 2 (IndF-7-Br), 3 (IndCl-7-Br), 7 (IndCl-7-Cl), and 9 (IndCl-7-F), which were observed to have 1.3~1.5-fold higher MBP expression compared to the vehicle-treated cells at 1 nM concentration. There were 5~6 wells/treatment group. n=5~6 independent experiments were performed.

FIGS. 1B and 1D show bar graphs quantifying the total number of OLs for each treatment group in Table 1. No significant differences in the total number of cells were observed between groups except for a reduction for IndF-7-Br (compound 2) and IndCl-7-Br (compound 3) at 10 nM. There were 5~6 wells/treatment group. n=5~6 independent experiments were performed.

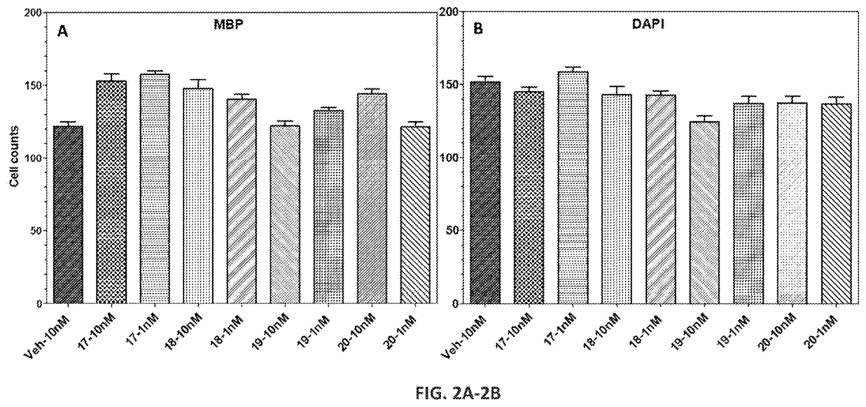

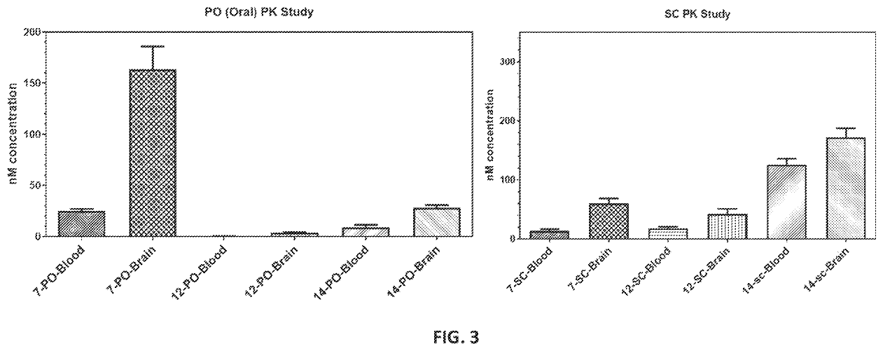

FIGS. 2A and 2B show the effects of treatment of the 6-substituted compounds 17-20 (IndCl-6-F, IndCl-6-F-2′-Me, IndCl-6-CI, and IndCl-6-Cl-2′-Me) on the expression of MBP⁺-OLs as an indication of OPC differentiation, as well as the total cell count (DAPI stain) as an indication of cell viability or proliferation. The cells were treated with the listed compounds (17-20) at both 1 nM and 10 nM. Compound 17 (IndCl-6-F) also shows a good MBP expression at 1 nM.

FIG. 2A shows bar graphs quantifying the number of MBP+OLs for each treatment group in Table 2. Analogs IndCl-6-F (compound 17) and IndCl-6-F-2′-Me (compound 18) showed a significant increase in the number of MBP+OLs that had an increase in the percentage of branched OLs, compared to vehicle-treated cells at both 1 nM and 10 nM. There was 5 wells/treatment group. n=5 independent experiments were performed.

FIG. 2B shows bar graphs quantifying the number of total cells for each treatment group in Table 2. No significant differences in the total number of cells were observed between groups at both 1 nM and 10 nM. There were 5 wells/treatment group. n=5 independent experiments were performed.

### Example 3. 2-Hour Single-Point Pharmacokinetic (PK) Study

The 2-Hour single-point pharmacokinetics for IndCl-7-Cl (compound 7), IndCl-7-Br-3′-OH (compound 12), and IndCl-7-Cl-3′-F (compound 14) was examined in (two and half month old C₅₇BL/6 female) mice after single subcutaneous (SC) injection or oral gavage (PO) in 0.1 mL at a dose 5 mg/kg for SC and 25 mg/kg for PO administration. The vehicle for SC administration was 0.1 mL 10% ethanol and 90% Miglyol 812N or 10% DMSO saline and for PO administration, 0.1 mL 2-hydroxypropyl-beta-cyclodextrin [HPCD]/40% aqueous solution MW ~1540. Two hours after injection blood and brain were collected and processed for quantification of the administered compound at the University of Illinois Metabolomics Core Facility by liquid chromatography-mass spectrometry (LC-MS/MS). The bar graphs indicate the analyzed concentration in the blood and the brain in nM units for each sample and each administered method. The brain uptake ratio over the blood of compound 7 (IndCl-7-C₁) in the PO and SC pharmacokinetic study is as high as ~7 and ~6, respectively.

Compound 7 (IndCl-7-Cl) shows selective high brain uptake in both PO and SC administration. The concentration of compound 7 (IndCl-7-C₁) in the brain after PO administration was markedly higher than that after SC injection.

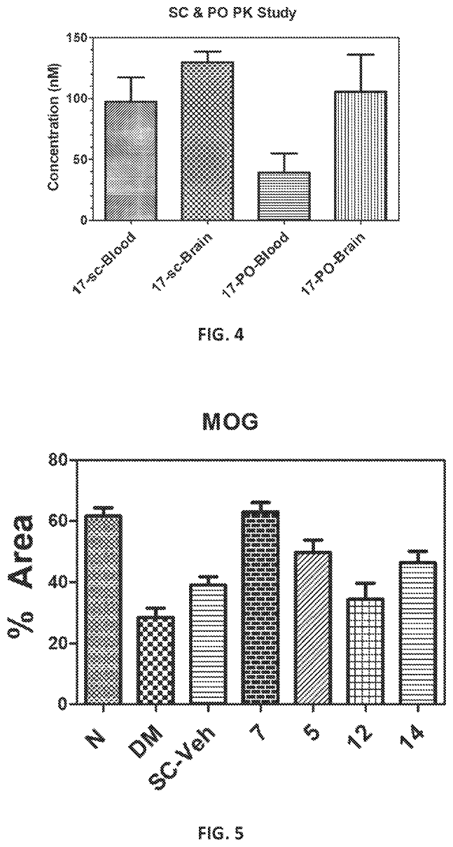

FIG. 4 shows a 2-hour single point pharmacokinetic study of compound 17. For subcutaneous (SC) injection, the compound made up as a homogeneous suspension in 10% ethanol and 90% Miglyol 812N (vehicle; Sasol) and a 0.1 mL dose was administered by SC injection at a dose of 5 mg/kg body weight. For PO administration, the compound was dissolved into 40% (2-Hydroxypropyl)-β-cyclodextrin (M_{w}~ 1540)(HP-CD) and administered via oral gavage in a volume of 0.1 mL at a dose of 25 mg/kg body weight.

After PO administration, the concentration of compound 17 (IndCl-6-F) in brain was 3-fold higher than the concentration in blood. (n=3).

### Example 4. Assessment of Myelin Recovery in the Cuprizone-Induced Demyelinated Mouse Model

The Cuprizone assay of demyelination-remyelination was conducted by the methods described previously (Moore et al. 2013; Crawford et al, 2009a,b). A group of animals was on normal diet (N groups). The remainder of the mice were divided into groups of 5 and all of these groups of mice were fed cuprizone diet at (0.2% cuprizone mixed in chow). After 6 weeks on cuprizone diet-induced demyelination, a group of 5 mice were perfused (DM group). The other groups of mice were switched to normal diet for 1.5 weeks to initiate remyelination and were treated with either (i) Vehicle (SC+V) or (ii) test compound at a dose of 5 mg/kg. A drug solution was prepared by combining 10% ethanol and 90% Miglyol oil for injection. After 1.5 weeks of normal diet-induced remyelination, the mice were perfused. Brains were collected, cryoprotected, embedded, cut, and IHC for myelin oligodendrocyte glycoproteins (MOG) was performed.

Cuprizone study results after 1.5-weeks treatment with the designated 7-substituted compounds via SC administration following 6 weeks cuprizone treatment of (C₅₇BL/6) female mice are shown in FIG. 5. The height of each bar graph represents level of myelination assessed by the expression of MOG in the white matter tracks of the Corpus Callosum area quantified by immunohistochemical analysis (IHC) after staining with a MOG antibody. N: Normal diet, DM: Demyelinated status after 6 weeks cuprizone treatment, SC-Veh: Spontaneous remyelination after injection with matrix in saline solution only (without compound; recovery is only about 40%). (Mice: ~2 months age, SC: Subcutaneous injection, n=5).

Compound 7 accelerates remyelination, reaching levels at or above the normal myelin level. Compounds 5 and 14 cause somewhat less remyelination; compound 12 does not accelerate remyelination.

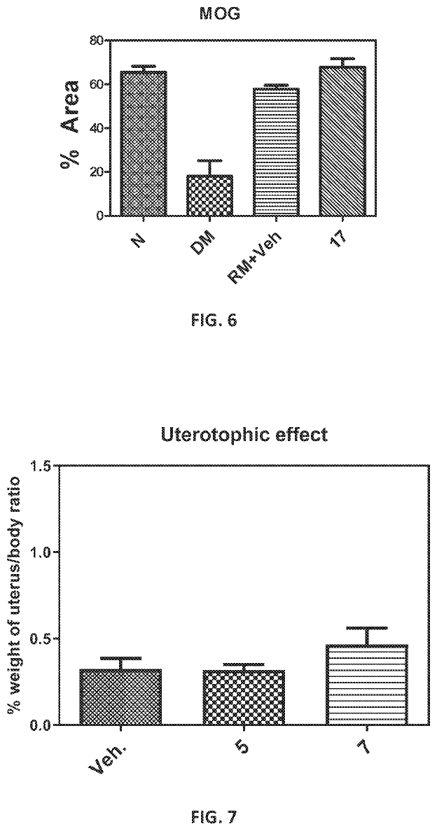

Cuprizone study results for the 3-weeks treatment with the 6-substituted compound 17 via SC administration following the 6 weeks of 0.2% cuprizone treatment of C57BL/6 female mice are shown in FIG. 6. The height of each bar graph represents level of myelination assessed by the expression of MOG in the white matter tracks of the Corpus Callosum area quantified by immunohistochemical analysis (IHC) after staining with a MOG antibody. N: Normal diet, DM: Demyelinated status after 6 weeks cuprizone treatment, SC-Veh: Spontaneous remyelination after injection with matrix in saline solution only (without compound; recovery is only about 40%). (Mice: ~2 months age, SC: Subcutaneous injection, n=5~6). 17: MOG staining represents to recover above the normal level by the compound 17. (Mice: ~2 months age, s.c.: Subcutaneous administration, n=5).

Compound 17 was observed to accelerate remyelination to reach a level at or above the normal level.

Uterotrophic effect of compounds 5 (IndI-7-Br) and 7 (IndCl-7-Cl) with the non-ovariectomized mice are shown in FIG. 7. The uterus was taken from the mice treated with compounds 5 and 7 from the recovery assessment study using the Cuprizone-induced demyelination model. Uterine weight for each compound is represented by % uterus/body weight. Compounds 5 and 7 did not show uterotrophic effects significantly different from mice treated with vehicle alone.

Estrogens increase uterine weight by acting primarily through ERα (Hewitt and Korach, 2003). In order to determine whether the analogues tested possessed ERα signaling properties, uterine weight was assessed. The compounds 5 (IndI-7-Br) and 7 (IndCl-7-C₁) did not stimulate the uterus weight significantly after daily treatment SC for three weeks at 5 mg/kg.

### Example 5. Experimental Autoimmune Encephalomyelitis (EAE)

EAE induction: Active EAE may be induced in eight-week-old female C₅₇BL/6 mice as previously described (Kumar et al., 2013; Hasselmann et al., 2017) (one of three representative EAE experiments). Briefly, mice receive two subcutaneous (s.c) injections, each consisting of MOG₃₅₋₅₅ peptide (Mimotopes, Clayton, Victoria, Australia) emulsified with M. butyricum-containing complete Freund's adjuvant (BD Difco, Franklin Lakes, NJ) supplemented with M. tuberculosis (BD Difco), followed by two intraperitoneal injections of Bordetella pertussis toxin (List Biological Laboratories, Campbell, CA). Mice may be monitored daily in accordance with standard EAE clinical disease scoring scale modified from Pettinelli and McFarlin (Pettinelli and McFarlin, 1981; Hasselmann et al., 2017). Animals are maintained in accordance with guidelines set by the National Institute of Health and as mandated by the University of California Riverside Office of Research Integrity and the Institutional Animal Care and Use Committee (IACUC) in compliance with the American Association for Laboratory Animal Science (AALAS).

Treatment: Test compounds may be dissolved in 10% ethanol and 90% Miglyol 812N (vehicle) (Cremer; Sasol, Germany). Positive control groups receive a 0.1 mL subcutaneous (s.c.) injection at 0.05 mg/kg/day E2 at EAE day 0 (preEAE). Therapeutic treatment (s.c) with vehicle and various ERβ ligands at 5 mg/kg/day may be initiated at EAE postinduction day 8 (postEAE; onset of clinical symptoms) and continued until day 30. Animals may be euthanized according to the 2013 AVMA Guidelines on Euthanasia and sacrificed on either on day 20-21 for flow cytometry, luminex analysis and immunohistochemistry or day 30 for electrophysiology after induction of disease.

Rotarod behavioral assay: Motor behavior may be tested up to two times per week for each mouse using a rotarod apparatus (Med Associates, Inc., St. Albans, VT). Briefly, animals are placed on a rotating horizontal cylinder for a maximum of 200 seconds. The amount of time the mouse remains walking on the cylinder without falling is recorded. Each mouse is tested on a speed of 3-30 rpm and given three trials for any given day. The three trials are averaged to report a single value for an individual mouse, and averages are then calculated for all animals within a given treatment group (Moore et al., 2014). The first two trial days prior to immunization serve as practice trials.

Histological Preparation of Tissues: Mice are deeply anesthetized by isoflurane (Piramal Healthcare) inhalation and perfused transcardially with phosphate buffered saline (PBS), followed by 10% formalin (Thermo Fisher Scientific) to fix tissues. Brains and spinal cords are dissected and post-fixed in 10% formalin (Thermo Fisher Scientific) for 24 hours, then cryoprotected in 30% sucrose (EMD Millipore, Darmstadt, Germany) for 48 hours and embedded in gelatin for sectioning. Embedded brains and spinal cords are then cut into 40-μm coronal sections using an HM525 NX cryostat (Thermo Fisher Scientific). Sections are collected serially and stored in PBS with 1% sodium azide at 4° C. until staining by immunohistochemistry, following a previously described protocol (Crawford et al., 2010; Moore et al., 2014).

Immunohistochemistry: Before histological staining, 40-μm free floating sections are thoroughly washed with PBS to remove residual sodium azide (Crawford et al., 2010). Sections are permeabilized with 0.3% Triton X-100 in 1×PBS and 15% normal goat serum (NGS). Myelination, gliosis and immune markers may be visualized by the following primary antibodies at a concentration of 1:500 unless otherwise noted: chicken anti-myelin basic protein (MBP; polyclonal, EMD Millipore, Darmstadt, Germany), chicken anti-glial fibrillary acidic protein (GFAP; EMD Millipore, Darmstadt), rat anti-cluster of differentiation 45 (CD45; clone 30-F11, BD Biosciences, San Diego, CA), mouse anti-ionized calcium-binding adapter molecule 1/allograft inflammatory factor-1 (Iba1/AIF1; clone 20A12.1, EMD Millipore, Darmstadt, Germany), goat anti-CXCL1 (R&D systems; Minneapolis, MN) at 1:250 and mouse anti-adenomatous polyposis coli (CC-1; clone CC-1, Genetex, Irvine, CA). Secondary staining may be performed using polyclonal fluorophore-conjugated antibodies from ThermoFisher Scientific at a concentration of 1:500 unless otherwise specified: goat anti-chicken Alexa Fluor® 555 (AF555), goat anti-rabbit Alexa Fluor®647 (AF647), donkey anti-chicken IgY Cy3 (EMD Millipore), goat anti-rat IgG AF647, goat anti-rabbit IgG Cy3 (EMD Millipore), goat anti-mouse IgG2b AF647 and rabbit anti-goat AF647. Nuclei may be counter stained with 4′,6-Diamidino-2-phenylindole (DAPI, 2 ng/ml; Molecular Probes) for 10 minutes after incubation with secondary antibodies, and sections mounted on glass slides, allowed to dry, and coverslipped with Fluoromount G mounting medium (Thermo Fisher Scientific) for imaging.

Splenocyte Isolation & Cytokine Analysis: On day 20-21 after induction of EAE, spleens are harvested prior to transcardial perfusion. Spleens are dissected from anesthetized mice and mechanically dissociated into a single cell suspension in cold RPMI 1640 supplemented with pyruvate, L-glutamine, and 10% fetal bovine serum (henceforth referred to as RPMI). Red blood cells are lysed by incubation with ACK buffer (VWR), washed, counted, and resuspended in RPMI for cytokine analysis. Splenocytes are then stimulated with 25 μg/ml MOG₃₅₋₅₅ and supernatants are collected 48 hours later (Khalaj et al., 2013; Moore et al., 2013). Levels of the anti-inflammatory cytokines: IL-10, IL-13, IL-4 and IL-5; pro-inflammatory cytokines IFNγ, IL-17, IL-1β TNFα, IL-6 and IL-2; and chemokines: CXCL1, CXCL10 may be determined by Cytokine Mouse Magnetic Panel for Luminex (Thermo Fisher Scientific; Waltham, MA) and run on the xMAP MAGPIX 100™ instrument (Luminex Corporation, Austin, Tx) according to manufacturer's instructions.

Transmission Electron Microscopy: Mice are perfused with PBS as above followed by paraformaldehyde/glutaraldehyde to preserve ultrastructure and Epon embedded as previously described (Crawford et al., 2010). Serial ultrathin sections of Epon-embedded CC may be stained with uranyl acetate-lead citrate for electron microscopy analysis. G-ratio may be measured using Fiji v1.0 Software (NIH).

Confocal Microscopy: Thoracic spinal cord dorsal and ventral column sections, as well as CC may be imaged using an Olympus BX61 confocal microscope (Olympus America Inc., Center Valley, PA) using a 10× and 40× objective. Z-stack projections were compiled using SlideBook 6 software (Intelligent Imaging Innovations, Inc., Denver, CO). Immunostaining may be quantified using unbiased stereology (Crawford et al., 2010). All images (RGB) may be Confocal converted to grayscale, split, and separated by color channel using imageJ version 2.2.0-rc-46/1.50 g (NIH). To avoid experimenter bias, auto-adjustment of brightness and contrast, as well as threshold of staining signal, may be carried out by ImageJ. MBP⁺, GFAP⁺, CD45⁺, Ibal⁺, CC1⁺, and CXCL1⁺, staining intensity is measured as percent area of positive immunoreactivity within the region of interest and intensity of signal determined by ImageJ.

Electrophysiology: To assess functional conductivity across the CC, electrophysiological recordings of compound action potentials (CAPs) may be measured as previously described (Crawford et al., 2009; Crawford et al., 2010). Coronal brain slices are prepared from adult (3 to 4 month old) C57BL/6 female mice. Briefly, mice are deeply anesthetized under isoflurane and decapitated. The brain is removed and submerged in partially frozen “slushy” solution of slicing buffer containing (in mM): 87 NaCl, 75 sucrose 2.5 KCl, 0.5 CaCl₂, 7 MgCl₂, 1.25 NaH₂PO₄, 25 NaHCO₃, 10 glucose, 1.3 ascorbic acid, 0.1 kynurenic acid, 2.0 pyruvate, and 3.5 MOPS, bubbled with 5% CO₂+95% O₂ (Lauderdale et al., 2015). Coronal slices (350 μm) are prepared using a Leica VT 1000S Vibratome (Bannockburn, IL) and subsequently incubated for 45 minutes at 35° C. in slicing buffer. Following incubation, slices are allowed to cool to room temperature for 15 minutes then transferred to ACSF (anterior cervical spine fixation) containing (in mM): 125 NaCl, 2.5 KCl, 2.5 CaCl₂), 1.3 MgCl₂, 1.25 NaH₂PO₄, 26.0 NaHCO₃, and 15 glucose, oxygenated with 5% CO₂+95% O₂. Slices are equilibrated in the standard ACSF for a minimum of 15-20 minutes prior to electrophysiological recordings. During electrophysiological recordings, slices are continuously perfused with oxygenated ACSF maintained at a flow rate of 1 mL/min. For recording CAPs, an Axon Digidata 1550 may be used with a Multiclamp 700B Amplifier and PClamp 10.4 Software (Molecular Devices, Sunnyvale, CA). Continuous recordings for CC conduction experiments may be low-pass filtered at 10 kHz and digitized at 200 kHz. All experiments are conducted at room temperature (24-26° C.). To stimulate the CC fiber tract, a concentric bipolar stimulating electrode (FHC Neural microTargeting Worldwide, Bowdoin, ME, USA) is placed approximately 1 mm away across from a recoding electrode (glass micropipette filled with ACSF) with a resistance of 1-3 MΩ. To elicit CAPs, an episodic stimulation protocol is created consisting of 8 consecutive sweeps, each 12 ms long, with a 5-see delay between sweeps and an immediate stimulus (0.01 ms duration) after the start of each sweep (Crawford et al., 2009a). Stimulus intensity is adjusted manually using an ISO-Flex stimulator (A.M.P.I). Standardized input-output plots are generated in current clamp mode for each slice by averaging at least 4 consecutive sweeps together to reduce the signal-to-noise ratio. Brain slices that exhibit near zero voltage even when stimulated with the maximal current are not included in the analysis. Electrophysiology data may be analyzed using Clampfit 10.4 software (Molecular Devices, Sunnyvale, CA) and OriginPro 2016 64 Bit (OriginLab Corporation)

Statistical Analysis: All statistics may be performed using Prism 6 software (GraphPad Software, La Jolla, CA). Differences in EAE clinical scores may be determined by two-way unbalanced ANOVA with Dunnett's multiple comparisons test (Hasselmann et al., 2017). Luminex data and immunohistochemistry data may be analyzed either by ordinary one-way ANOVA with Dunnett's multiple comparisons test if data satisfied assumptions of normal distribution (D'Agostino & Pearson omnibus normality test) and equal variances among all groups or Kruskal Wallis with Dunn's multiple comparisons test. CAP recording analysis may be carried out per previously published work (Crawford et al., 2009b; Moore et al., 2014) using Clampfit 10.4 software (Molecular Devices, Sunnyvale, CA), OriginPro 2016 64 Bit (OriginLab Corporation) and GraphPad Prism 6 (GraphPad Software). The averaged mean amplitude is compared using one-way ANOVA with post hoc tests using Tukey's multiple comparison test.

All data is presented as mean±SEM for two independent experiments. Differences are considered significant at * p≤0.05, ** p≤0.01, and *** p≤0.001, **** p≤0.0001.

While the present invention is susceptible to various modifications and alternative forms, exemplary embodiments thereof are shown by way of example in the drawings and are herein described in detail. It should be understood, however, that the description of exemplary embodiments is not intended to limit the invention to the particular forms disclosed, but on the contrary, the intention is to cover all modifications, equivalents and alternatives falling within the spirit and scope of the invention as defined by the embodiments above and the claims below. Reference should therefore be made to the embodiments above and claims below for interpreting the scope of the invention.

### REFERENCES

Banks E, Canfell K (2009) Invited Commentary: Hormone therapy risks and benefits—The Women's Health Initiative findings and the postmenopausal estrogen timing hypothesis. Am J Epidemiol 170:24-28.
Burns K A, Korach K S (2012) Estrogen receptors and human disease: an update. Arch Toxicol 86:1491-1504.
Crawford D K, Mangiardi M, Tiwari-Woodruff S K (2009a) Assaying the functional effects of demyelination and remyelination: revisiting field potential recordings. J Neurosci Methods 182:25-33.
Crawford D K, Mangiardi M, Xia X, Lopez-Valdes H E, Tiwari-Woodruff S K (2009b) Functional recovery of callosal axons following demyelination: a critical window. Neuroscience 164:1407-1421.
Crawford D K, Mangiardi M, Song B, Patel R, Du S, Sofroniew M V, Voskuhl R R, Tiwari-Woodruff S K (2010) Oestrogen receptor beta ligand: a novel treatment to enhance endogenous functional remyelination. Brain 133:2999-3016.
Cua D J, Hinton D R, Stohlman S A (1995) Self-antigen-induced Th2 responses in experimental allergic encephalomyelitis (EAE)-resistant mice. Th2-mediated suppression of autoimmune disease. J Immunol 155:4052-4059.
De Angelis M, Stossi F, Carlson K A, Katzenellenbogen B S, Katzenellenbogen J A (2005) Indazole Estrogens: Highly Selective Ligands for the Estrogen Receptor β. Journal of Medicinal Chemistry 48:1132-1144.
Hasselmann J P C, Karim H, Khalaj A J, Ghosh S, Tiwari-Woodruff SK (2017) Consistent induction of chronic experimental autoimmune encephalomyelitis in C₅₇BL/6 mice for the longitudinal study of pathology and repair. J Neurosci Methods 284:71-84.
Hewitt S C, Korach K S (2003) Oestrogen receptor knockout mice: roles for oestrogen receptors alpha and beta in reproductive tissues. Reproduction 125:143-149.
Jansson L, Holmdahl R (1998) Estrogen-mediated immunosuppression in autoimmune diseases. Inflamm Res 47:290-301.
Karim, H., Kim, S. H., Lauderdale, K., Lapato A S, Atkinson K, Yasui N, Yamate-Morgan H, Sekyi M, Katzenellenbogen J A, Tiwari-Woodruff S K (2019) Analogues of ERβ ligand chloroindazole exert immunomodulatory and remyelinating effects in a mouse model of multiple sclerosis. Sci Rep 9, 503.
Khalaj A J (2016) Nudging oligodendrocyte intrinsic signaling to remyelinate and repair: Estrogen receptor ligand effects. 160:43-52.
Khalaj A J, Yoon J, Nakai J, Winchester Z, Moore S M, Yoo T, Martinez-Torres L, Kumar S, Itoh N, Tiwari-Woodruff S K (2013) Estrogen receptor (ER) beta expression in oligodendrocytes is required for attenuation of clinical disease by an ERbeta ligand. Proc Natl Acad Sci USA 110:19125-19130.
Kim S, Liva S M, Dalal M A, Verity M A, Voskuhl R R (1999) Estriol ameliorates autoimmune demyelinating disease: implications for multiple sclerosis. Neurology 52:1230-1238.
Kumar S, Patel R, Moore S, Crawford D K, Suwanna N, Mangiardi M, Tiwari-Woodruff SK (2013) Estrogen receptor beta ligand therapy activates PI3K/Akt/mTOR signaling in oligodendrocytes and promotes remyelination in a mouse model of multiple sclerosis. Neurobiol Dis 56:131-144.
Lauderdale K, Murphy T, Tung T, Davila D, Binder D K, Fiacco T A (2015) Osmotic Edema Rapidly Increases Neuronal Excitability Through Activation of NMDA Receptor-Dependent Slow Inward Currents in Juvenile and Adult Hippocampus. ASN Neuro 7.
Monnerie H, Romer M, Jensen B K, Millar J S, Jordan-Sciutto K L, Kim S F, Grinspan J B (2017) Reduced sterol regulatory element-binding protein (SREBP) processing through site-1 protease (S1P) inhibition alters oligodendrocyte differentiation in vitro. J Neurochem 140:53-67.
Moore S, Khalaj A J, Yoon J, Patel R, Hannsun G, Yoo T, Sasidhar M, Martinez-Torres L, Hayardeny L, Tiwari-Woodruff S K (2013) Therapeutic laquinimod treatment decreases inflammation, initiates axon remyelination, and improves motor deficit in a mouse model of multiple sclerosis. Brain Behav 3:664-682.
Moore S M, Khalaj A J, Kumar S, Winchester Z, Yoon J, Yoo T, Martinez-Torres L, Yasui N, Katzenellenbogen J A, Tiwari-Woodruff S K (2014) Multiple functional therapeutic effects of the estrogen receptor beta agonist indazole-Cl in a mouse model of multiple sclerosis. Proc Natl Acad Sci USA 111: 18061-18066.
Nicot A (2009) Gender and sex hormones in multiple sclerosis pathology and therapy. Front Biosci 14:4477-4515.
Pettinelli C B, McFarlin D E (1981) Adoptive transfer of experimental allergic encephalomyelitis in SJL/J mice after in vitro activation of lymph node cells by myelin basic protein: requirement for Lyt 1+2-T lymphocytes. Journal of immunology 127:1420-1423.
Tiwari-Woodruff S K, Buznikov A G, Vu T Q, Micevych P E, Chen K, Kornblum H I, Bronstein J M (2001) OSP/claudin-11 forms a complex with a novel member of the tetraspanin super family and beta1 integrin and regulates proliferation and migration of oligodendrocytes. J Cell Biol 153:295-305.
Tiwari-Woodruff S, Beltran-Parrazal L, Charles A, Keck T, Vu T, Bronstein J (2006a) K+channel KV3.1 associates with OSP/claudin-11 and regulates oligodendrocyte development. Am J Physiol Cell Physiol 291: C687-698.
Tiwari-Woodruff S, Morales L, Loo K, Liu H, Peterson C, Voskuhl R (2006b) Treatment with an estrogen receptor a ligand is both anti-inflammatory and neuroprotective in experimental autoimmune encephalomyelitis. JOURNAL OF NEUROCHEMISTRY 96:23-23.

We claim:

1. A compound of formula (I), or a pharmaceutically acceptable salt thereof, 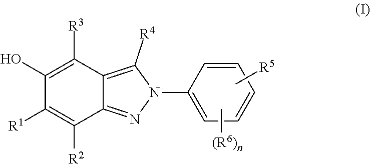
wherein:
R¹ and R² are independently hydrogen, halogen, C₁₋₄alkyl, C₁₋₄fluoroalkyl, C₂₋₄alkenyl, cyano, OH, —OC₁₋₄alkyl, or —OC₁₋₄fluoroalkyl, provided that at least one of R¹ and R² is not hydrogen;
R³ is hydrogen, halogen, or C₁₋₄alkyl;
R⁴ is hydrogen, halogen, C₁₋₄alkyl, C₁₋₄fluoroalkyl, or C₂₋₄alkenyl;
R⁵ is OH, wherein R⁵ is substituted at either the meta or para position;
R⁶, at each occurrence, is independently halogen, C₁₋₄alkyl, C₁₋₄fluoroalkyl, C₂₋₄alkenyl, cyano, OH, —OC₁₋₄alkyl, or —OC₁₋₄fluoroalkyl; and
n is 0, 1, or 2.

2. The compound of claim 1, or a pharmaceutically acceptable salt thereof, wherein R¹ is halogen, C₁₋₄alkyl, C₁₋₄fluoroalkyl, C₂₋₄alkenyl, cyano, —OC₁₋₄alkyl, or —OC₁₋₄fluoroalkyl.

3. The compound of claim 1, or a pharmaceutically acceptable salt thereof, wherein R¹ is hydrogen.

4. The compound of any of claims 1-3, or a pharmaceutically acceptable salt thereof, wherein R² is halogen, C₁₋₄alkyl, C₁₋₄fluoroalkyl, C₂₋₄alkenyl, cyano, OH, —OC₁₋₄alkyl, or —OC₁₋₄fluoroalkyl.

5. The compound of claim 2, or a pharmaceutically acceptable salt thereof, wherein R² is hydrogen.

6. The compound of any of claims 1-5, or a pharmaceutically acceptable salt thereof, wherein R³ is hydrogen.

7. The compound of any of claims 1-6, or a pharmaceutically acceptable salt thereof, wherein R⁴ is halogen.

8. The compound of any of claims 1-6, or a pharmaceutically acceptable salt thereof, wherein R⁴ is hydrogen.

9. The compound of any of claims 1-8, or a pharmaceutically acceptable salt thereof, wherein R⁵ is substituted at the meta position.

10. The compound of any of claims 1-8, or a pharmaceutically acceptable salt thereof, wherein R⁵ is substituted at the para position.

11. The compound of any of claims 1-10, or a pharmaceutically acceptable salt thereof, wherein n is 1.

12. The compound of claim 11, or a pharmaceutically acceptable salt thereof, wherein the compound has formula (II):

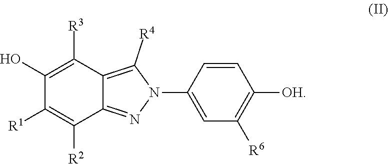

13. The compound of claim 11, or a pharmaceutically acceptable salt thereof, wherein the compound has formula (III):

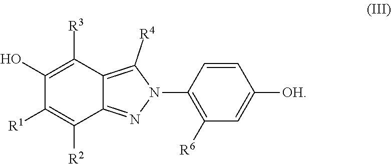

14. The compound of any of claims 1-13, or a pharmaceutically acceptable salt thereof, wherein R⁶, at each occurrence, is independently halogen or C₁₋₄alkyl.

15. The compound of any of claims 1-10, or a pharmaceutically acceptable salt thereof, wherein n is 0.

16. The compound of claim 15, or a pharmaceutically acceptable salt thereof, wherein the compound has formula (IV):

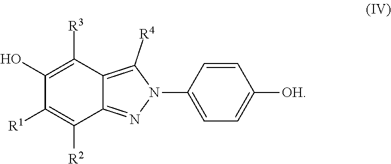

17. The compound of claim 15, or a pharmaceutically acceptable salt thereof, wherein the compound has formula (V):

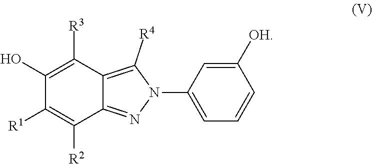

18. The compound of claim 1 selected from the group consisting of:
7-bromo-2-(4-hydroxyphenyl)-2H-indazol-5-ol;
7-bromo-3-fluoro-2-(4-hydroxyphenyl)-2H-indazol-5-ol;
7-bromo-3-chloro-2-(4-hydroxyphenyl)-2H-indazol-5-ol;
3,7-dibromo-2-(4-hydroxyphenyl)-2H-indazol-5-ol;
7-bromo-2-(4-hydroxyphenyl)-3-iodo-2H-indazol-5-ol;
7-chloro-2-(4-hydroxyphenyl)-2H-indazol-5-ol;
3,7-dichloro-2-(4-hydroxyphenyl)-2H-indazol-5-ol;
7-fluoro-2-(4-hydroxyphenyl)-2H-indazol-5-ol;
3-chloro-7-fluoro-2-(4-hydroxyphenyl)-2H-indazol-5-ol;
7-methyl-2-(4-hydroxyphenyl)-2H-indazol-5-ol;
3-chloro-7-methyl-2-(4-hydroxyphenyl)-2H-indazol-5-ol;
7-bromo-3-chloro-2-(3-hydroxyphenyl)-2H-indazol-5-ol;
7-chloro-2-(3-fluoro-4-hydroxyphenyl)-2H-indazol-5-ol;
3,7-dichloro-2-(3-fluoro-4-hydroxyphenyl)-2H-indazol-5-ol;
7-chloro-2-(2-fluoro-4-hydroxyphenyl)-2H-indazol-5-ol;
3,7-dichloro-2-(2-fluoro-4-hydroxyphenyl)-2H-indazol-5-ol;
3-chloro-6-fluoro-2-(4-hydroxyphenyl)-2H-indazol-5-ol;
3-chloro-6-fluoro-2-(4-hydroxy-2-methylphenyl)-2H-indazol-5-ol;
3,6-dichloro-2-(4-hydroxyphenyl)-2H-indazol-5-ol;
3,6-dichloro-2-(4-hydroxy-2-methylphenyl)-2H-indazol-5-ol;
3-chloro-2-(4-hydroxyphenyl)-2H-indazole-5,7-diol; and
3-chloro-2-(4-hydroxy-2-methylphenyl)-2H-indazole-5,7-diol;
or a pharmaceutically acceptable salt thereof.

19. A pharmaceutical composition comprising the compound of any of claims 1-18, or a pharmaceutically acceptable salt thereof, and a pharmaceutically acceptable carrier.

20. A method of treating a demyelinating disease comprising, administering to a subject in need thereof, a therapeutically effective amount of the compound of any of claims 1-18, or a pharmaceutically acceptable salt thereof, or the pharmaceutical composition of claim 19.

21. The method of claim 20, wherein the demyelinating disease is multiple sclerosis.

22. The method of claim 21, wherein the multiple sclerosis is primary progressive multiple sclerosis, relapsing-remitting multiple sclerosis, secondary progressive multiple sclerosis, or progressive relapsing multiple sclerosis.

23. A method of promoting remyelination of demyelinated axons comprising administering to a subject in need thereof a therapeutically effective amount of the compound of any of claims 1-18, or a pharmaceutically acceptable salt thereof, or the pharmaceutical composition of claim 19.

24. A method of differentiating oligodendrocyte progenitor cells comprising administering to a subject in need thereof a therapeutically effective amount of the compound of any of claims 1-18, or a pharmaceutically acceptable salt thereof, or the pharmaceutical composition of claim 19.

25. A method of treating endometriosis comprising administering, to a subject in need thereof, a therapeutically effective amount of the compound of any of claims 1-18, or a pharmaceutically acceptable salt thereof, or the pharmaceutical composition of claim 19.

26. The compound of any of claims 1-18, or a pharmaceutically acceptable salt thereof, or the pharmaceutical composition of claim 19, for use in the treatment of demyelinating disease, or in the promotion of remyelination of demyelinated axons, or in the differentiation of oligodendrocyte progenitor cells, or in the treatment of endometriosis.

27. The use of the compound of any of claims 1-18, or a pharmaceutically acceptable salt thereof, or the pharmaceutical composition of claim 19, in the manufacture of a medicament for the treatment of demyelinating disease, or for the promotion of remyelination of demyelinated axons, or for the differentiation of oligodendrocyte progenitor cells, or for the treatment of endometriosis.

28. A kit comprising the compound of any of claims 1-18, or a pharmaceutically acceptable salt thereof, or the pharmaceutical composition of claim 19, and instructions for use thereof.
